(19) United States
(12) Patent Application Publication
(10) Pub. No.: US 2024/0282402 A1
(43) Pub. Date: Aug. 22, 2024

(54) SYSTEM AND METHOD FOR ACCELERATING WHOLE CELL SIMULATIONS

(71) Applicant: RAMOT AT TEL-AVIV UNIVERSITY LTD., Tel Aviv (IL)

(72) Inventors: Tamir TULLER, Tel Aviv (IL); David SHALLOM, Tel Aviv (IL); Shlomo WEISS, Tel Aviv (IL); Danny NAIGER, Tel Aviv (IL)

(73) Assignee: RAMOT AT TEL-AVIV UNIVERSITY LTD., Tel Aviv (IL)

(21) Appl. No.: 18/641,391

(22) Filed: Apr. 21, 2024

Related U.S. Application Data
(63) Continuation of application No. PCT/IL2022/051132, filed on Oct. 27, 2022.
(60) Provisional application No. 63/272,865, filed on Oct. 28, 2021.

(51) Int. Cl. G16B 5/00

(52) U.S. Cl. CPC G16B 5/00

(57) ABSTRACT

A system and method of whole-cell process simulations may include: using at least one first hardware module, to produce a first predicted value, representing an expected behavior of a plurality of resource entities in a simulated biological cell; using at least one second hardware module, to produce a second predicted value, representing an interaction of a plurality of client entities with at least one of the plurality of resource entities in the simulated biological cell; using at least one third hardware module, to produce a third predicted value, representing an arbitration of interactions between the plurality of resource entities and the plurality of client entities in the simulated biological cell; and producing a simulated value of a product of a natural process in the simulated biological cell, based on said first, second and third predicted values.

### CROSS REFERENCE TO RELATED APPLICATIONS

This application is a ByPass Continuation of PCT Patent Application No. PCT/IL2022/051132 having International filing date of Oct. 27, 2022, which claims the benefit of priority of U.S. Provisional Patent Application No. 63/272,865, filed on Oct. 28, 2021 titled “SYSTEM AND METHOD FOR ACCELERATING WHOLE CELL SIMULATIONS”. The contents of the above applications are all incorporated by reference as if fully set forth herein in their entirety.

### FIELD OF THE INVENTION

The present invention relates generally to simulation of biological processes. More specifically, the present invention relates to systems and methods for accelerating whole-cell process simulations.

### BACKGROUND OF THE INVENTION

Biophysical models of intracellular processes such as gene expression have been used in recent years for studying numerous questions related to all biomedical disciplines. The more advanced models in the field consider the competition′ of “clients entities” in the cell over resource entities. In recent years, we understand that without considering this aspect of competition, the models usually provide significantly biased prediction. However, currently available systems and methods for whole-cell biological process simulation perform poorly due to computational constraints.

### SUMMARY OF THE INVENTION

Taking as an example the biological process of mRNA translation, by which ribosomes are attached to mRNA strands for the purpose of producing protein: In this example, the mRNA strands may be regarded as clients, which compete over the limited resources of ribosomes. Since a typical cell includes thousands of mRNAs and ribosomes, the simulation of such a process is computationally challenging and cannot be parallelized easily, in a manner that genuinely simulates the behaviour of cellular entities, and the relations therebetween. For example, as the state of each mRNA molecule depends on the global assignment of ribosomes to all mRNA molecules in a cell, a software-based application for simulating mRNA molecule translation should operate in a synchronized manner. Such constraints requires large synchronization overheads that degrade the performance of processor-based implementations that accommodate large amounts of parallel threads. This is specifically challenging when various sets of parameters of the models are studied or optimized as in the case of synthetics biology where the aim is to find a set of modifications in the cell that will optimize a certain objective which is affected by a large pool of factors in the cell. In such cases, the optimization process may often be prohibitively long.

There is therefore a need for a system and method capable of accelerated whole cell simulation of biological processes that would overcome the drawbacks described above.

Embodiments of the invention may facilitate a novel approach for addressing this challenge by using dedicated hardware, designed specifically to simulate such processes. As a non-limiting example, the description focuses on the biological process of mRNA translation. However, it may be appreciated that embodiments of the invention may be modified to simulate other such processes, as discussed herein (e.g., in relation to FIG. 23).

Embodiments of the invention may include a system and method of whole-cell process simulation by one or more dedicated, hardware-implemented electrical circuits. The term “dedicated” may be used in this context in a sense that the hardware-implemented electrical circuits may be uniquely designed to expedite simulation of an underlying biological process, and therefore may not be implemented, or entirely implemented by a generic combination of hardware and software such as a software executed by a generic computer. For example, hardware-implemented electrical circuits of the present invention may be implemented, at least in part by programmable logic on a hardware electrical circuit, such as a Field Programmable Gate Array (FPGA) chip or an Application Specific Integrated Circuit (ASIC) chip.

Embodiments of the method may include using a plurality of hardware-implemented electrical circuits, referred to herein as resource modules, each corresponding to at least one simulated resource entity in a simulated biological cell. The plurality of resource hardware modules may be configured to predict a respective plurality of resource behaviour values. Each resource behaviour value may represent an aspect of behaviour of the at least one corresponding simulated resource entity in a simulated biological cell.

Embodiments of the method may further include using a plurality of hardware-implemented electrical circuits, referred to herein as client hardware modules, each corresponding to a simulated client entity in the simulated biological cell. The plurality of client hardware modules may be configured to predict a respective plurality of interaction values. Each interaction value may represent an aspect of interaction of the corresponding simulated client entity with at least one of said simulated resource entities.

As elaborated herein, the one or more hardware-implemented electrical circuits may be configured to calculate a simulated product value, representing a product of a biological process in the simulated biological cell, based on said interaction values and resource behaviour values.

Additionally, or alternatively, the one or more hardware-implemented electrical circuits may further include at least one electrical circuit referred to herein as an arbitration hardware module. Embodiments of the method may include using the at least one arbitration hardware module to allocate one or more resource hardware modules of the plurality of resource hardware modules to at least one client hardware module of the plurality of client hardware modules. The at least one arbitration hardware module may be configured to predict one or more arbitration values based on said allocation. Each arbitration value may represent an aspect of allocation of simulated resource entities to simulated client entities in the simulated biological cell.

According to some embodiments, the one or more hardware-implemented electrical circuits may be configured to calculate the simulated product value further based on the one or more arbitration values.

According to some embodiments, at least one of the resource hardware modules, client hardware modules and arbitration hardware modules may be implemented, at least in part, as programmable logic on a hardware electrical circuit, such as an FPGA chip or an ASIC chip.

According to some embodiments, the simulated biological process may be a process of mRNA translation, and subsequent production of simulated proteins. In such embodiments, the simulated resource entities may be simulated ribosomes of the simulated biological cell, and the simulated client entities may be simulated mRNA strands of the simulated biological cell.

According to some embodiments, the resource behaviour value may be, for example a resource status, indicating whether a corresponding simulated ribosome is either (i) currently associated to a pool of free ribosomes, or (ii) allocated to a simulated mRNA strand of the simulated biological cell.

Additionally, or alternatively, the resource behaviour value may be for example, a duration of translation of at least one simulated codon or codon type by the corresponding simulated ribosome; a duration of the corresponding simulated ribosome to process a predetermined number of simulated codons; an initiation rate representing a time it takes for the corresponding simulated ribosome to initiate translation of a simulated mRNA strand; a ribosome footprint, representing a number of simulated codons that the corresponding simulated ribosome may handle concurrently; and a diffusion delay, representing a time it takes for the corresponding simulated ribosome, after finishing translation of one mRNA strand, to become available for translating another simulated mRNA strand.

Additionally, or alternatively, the interaction value may represent a state of activity of one or more simulated ribosomes allocated to the corresponding simulated mRNA strand, where said state of activity may be (i) an inactive state, and (ii) an active state, in which translation of a simulated codon may be currently performed.

Additionally, or alternatively, the interaction value may further be a number of simulated ribosomes that may be applied to the corresponding simulated mRNA strand; a number of active simulated ribosomes, that may be currently performing translation of the corresponding simulated mRNA strand; a location of one or more simulated ribosomes on the corresponding simulated mRNA strand; and a codon index, representing a codon that may be being translated by a simulated ribosome on the corresponding simulated mRNA strand.

According to some embodiments, the arbitration values may represent aspects of allocation of simulated ribosomes to simulated mRNA strands in the simulated biological cell. Such aspects may include, for example an overall number of simulated ribosomes in the simulated biological cell; an overall number of simulated mRNA strands in the simulated biological cell; a number of ribosomes in the simulated biological cell that may be available for mRNA translation; a number of simulated mRNA strands that may be currently allocated to simulated ribosomes; a number of simulated mRNA strands that may be currently being translated by allocated simulated ribosomes; and a number of simulated ribosomes that may be allocated to each simulated mRNA strand.

According to some embodiments, the biological process may be a process of translation of the simulated mRNA strands by the plurality of simulated ribosomes, and the simulated product value may be a simulated quantity of protein molecules, produced in the process of mRNA translation.

Additionally or alternatively, the biological process may be a process of gene transcription. Accordingly, the plurality of resource hardware modules may represent simulated RNA polymerase molecules, and the plurality of client hardware modules may represent simulated genes in the simulated biological cell.

According to some embodiments, the at least one arbitration hardware module may be configured to allocate the one or more resource hardware modules to the plurality of client hardware modules with uniform probability.

According to some embodiments, the plurality of client hardware modules may include at least one client hardware module of a first client type, referred to herein as an “iterative” client, and one or more second client hardware modules of a second client type, referred to herein as a “parallel” client. In such embodiments, predicting an interaction value may include: (i) using the at least one client hardware module of the first client type to identify a subset of simulated client entities in the simulated biological cell, characterized by a first desired objective; and (ii) using the one or more client hardware modules of the second client type, to select a simulated client entity in the simulated biological cell, characterized by a predefined synthetic biological objective.

Embodiments of the invention may include a system for whole-cell process simulation. Embodiments of the system may include a plurality of hardware-implemented electrical circuits, referred to herein as resource hardware modules. Each resource hardware module may represent at least one simulated resource entity in a simulated biological cell, and configured to predict respective resource behaviour values. Each resource behaviour value represents an aspect of behaviour of the at least one corresponding simulated resource entity. Embodiments of the system may further include a plurality of hardware-implemented electrical circuits, referred to herein as client hardware modules. Each client hardware module may represent a simulated client entity in the simulated biological cell, and configured to predict respective interaction values. Each interaction value represents an aspect of interaction of the corresponding simulated client entity with at least one of said simulated resource entities.

According to some embodiments, the plurality of resource hardware modules and the plurality of client hardware modules may be implemented, at least in part as programmable logic on a dedicated hardware electrical circuit such as an FPGA chip or an ASIC chip. Embodiments of the system may further include at least one processor embedded in the dedicated hardware electrical circuit (FPGA or ASIC chip). The at least one processor may be configured to calculate a simulated product value, representing a product of a biological process in the simulated biological cell, based on the interaction values and resource behaviour values.

Embodiments of the system may further include at least one hardware-implemented electrical circuit, referred to herein as an arbitration hardware module. The at least one Arbitration hardware module may be configured to allocate one or more resource hardware modules of the plurality of resource hardware modules to at least one client hardware module of the plurality of client hardware modules. According to some embodiments, the at least one processor may be further configured to: predict one or more arbitration values based on said allocation, wherein each arbitration value representing an aspect of allocation of simulated resource entities to simulated client entities in the simulated biological cell; and calculate the simulated product value further based on said one or more arbitration values.

According to some embodiments, at least one of the resource hardware modules, client hardware modules and arbitration hardware modules may be at least partially implemented as programmable logic on a hardware electrical circuit, such as an FPGA chip or an ASIC chip.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a flow graph which depicts an exemplary application of a system (also referred to herein as a model) for simulating whole-cell biological processes, according to some embodiments of the invention;

FIG. 2 is a schematic illustration for the biophysical process of mRNA translation;

FIG. 3 is a High-level block diagram depicting hardware modules of a system for performing whole-cell process simulation, according to embodiments of the present invention. The dashed lined components are for synchronizing the mRNA modules in the iterative model and are not present in the parallel model;

FIG. 4 is a schematic illustration of an arbiter module, according to embodiments of the present invention.

FIG. 5 is a schematic block diagram of a parallel mRNA module and its local, allocated hardware ribosomes, according to embodiments of the present invention;

FIG. 6 is a schematic hardware state machine representing states of a simulated ribosome, according to embodiments of the present invention;

FIG. 7 is a schematic block diagram depicting an exemplary implementation of an iterative client (e.g., mRNA) module, according to embodiments of the present invention;

FIG. 8 is a schematic block diagram depicting an example for implementation of a system for simulating whole-cell biological processes according to embodiments of the present invention;

FIG. 9 is a schematic flow graph illustrating the order in which operations are carried out by the system for simulating whole-cell biological processes, according to embodiments of the present invention;

FIG. 10 is a graph depicting the number of consecutive Bernoulli (with p=1/512) experiments needed for at least two success events with miss probability lower than 1e-5, according to embodiments of the present invention;

FIG. 11 presents 16 bit matrices for generating random numbers in hardware, according to embodiments of the present invention;

FIG. 12 is a graph depicting utilization of allocated hardware ribosomes using different arbiters and different allocation methods, according to embodiments of the present invention;

FIG. 13 is a graph depicting local mRNA's data arbiter size in LUTs as a function of number of hardware ribosomes, the number of mRNAs that are free to receive new ribosomes as a function of time, according to embodiments of the present invention;

FIG. 14 is a schematic diagram showing storage of simulated codons' data, according to embodiments of the present invention;

FIG. 15 is a block diagram depicting an example for implementation of a synchronization mechanism, according to some embodiments of the invention;

FIG. 16 is a graph showing the number of active ribosomes on each mRNA molecule as function of time in the initial model for selected mRNA molecules;

FIG. 17 is a block diagram depicting connections between resource modules (hardware ribosomes) and a global data arbiter, according to some embodiments of the invention;

FIG. 18 is a block diagram depicting an example of a state machine of a resource (e.g., ribosome) module, according to some embodiments of the invention;

FIG. 19 is a block diagram depicting another example of a state machine of a resource (e.g., ribosome) module, according to some embodiments of the invention;

FIG. 20 is a block diagram depicting HDL modules' hierarchy of the parallel mRNA module, according to some embodiments of the invention;

FIG. 21 is a block diagram depicting HDL modules' hierarchy of the configurable iterative mRNA module, according to some embodiments of the invention;

FIG. 22 is a high-level block diagram depicting an example of a system for whole-cell process simulation, according to some embodiments of the invention;

FIG. 23 is a high-level block diagram depicting another example of a system for whole-cell process simulation, according to some embodiments of the invention; and

FIG. 24 is a flow diagram depicting an example of a method of whole-cell process simulation by one or more dedicated, hardware-implemented electrical circuits, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Embodiments of the invention that are described herein demonstrate a new approach for tackling this challenge based on the design of a dedicated hardware that can yield an optimization process which is orders of magnitudes faster.

While there exist few previous studies describing very small analogous circuits that are inspired by biological phenomena, no previous study included digital parallel whole cell dedicated hardware. Embodiments of the present invention may utilize at least one hardware circuit or System on Chip (SoC) module, such as a Field Programmable Gate Array (FPGA) chip or Application-Specific Integrated Circuit (ASIC) chip for the purpose of whole-cell biological process modelling or simulation.

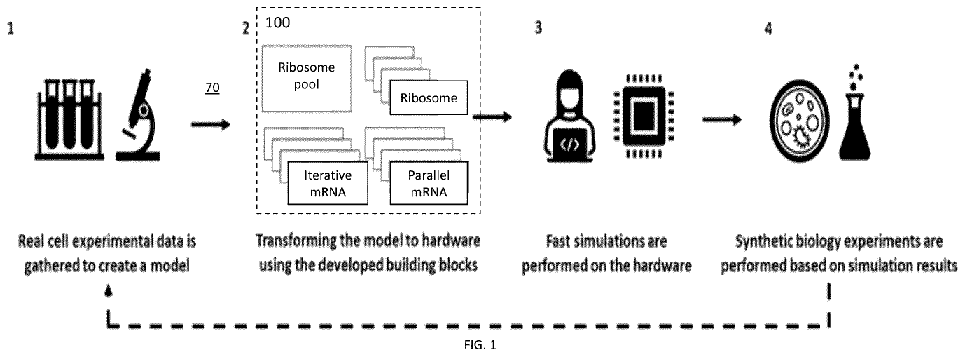

Reference is now made to FIG. 1 which is a flow diagram depicting an exemplary application of a system 100 (also referred to herein as a model 100) for simulating whole-cell biological processes, according to some embodiments of the invention. As shown in step (1), large scale experimental biological measurements and data are obtained, based on which model 100 may be configured. Such biological data may include, for example, biological coding sequences, cellular mRNA levels, ribosomal densities, and the like. As shown in step (2), the simulative model is transformed into hardware modules, which integrate the obtained biological measurements, and combine the building blocks described herein. As shown in step (3), consecutive fast runs of the model 100 in hardware may be executed, using various parameters or values of the biological data. As shown in step (3), based on the results of model 100 executions, synthetic biology experiments may be performed, e.g., in order to determine optimal parameters for predefined or desired phenotypic or proteomic results. The entire process can then be repeated based on new experimental observations, as depicted by the broken arrow line.

According to embodiments of the present invention, model or system 100 may consider various biological properties, that may be defined according to an underlying task.

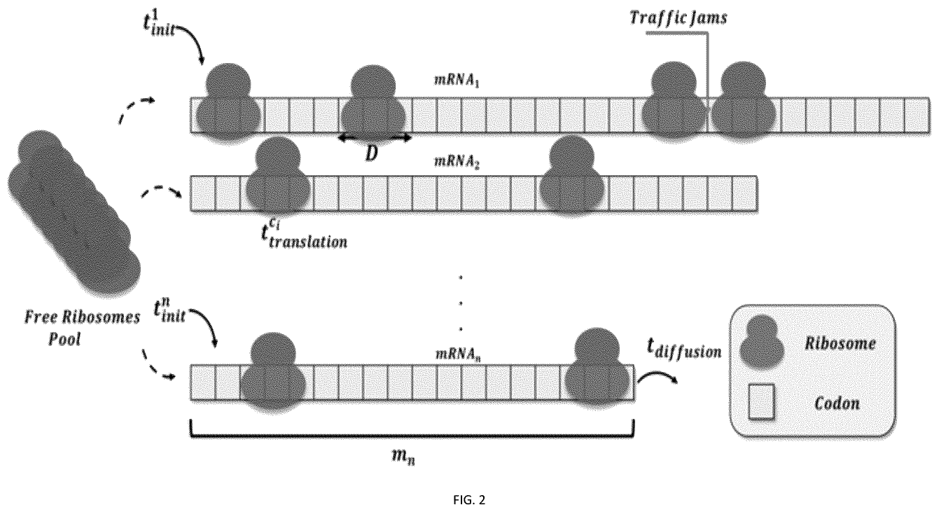

Reference is now made FIG. 2 which is a schematic illustration depicting a biophysical process of mRNA translation. Pertaining to the example of FIG. 2, a system 100 configured to model or simulate mRNA translation according to embodiments of the invention may receive one or more biological parameter data elements 70, representing properties or characteristics of the underlying biological process (e.g., mRNA translation). Biological parameter data elements 70 may also be referred to herein as biological parameters 70 or biological facts 70.

According to some embodiments, system 100 may be configured to consider biological parameters 70 or biological facts 70 during the process of whole-cell simulation.

In the example of mRNA translation, biological parameters 70 may include, for example, a number of ribosomes in the simulated cell. This corresponds to the fact that different mRNA molecules compete for the same pool of ribosomes, i.e., a limited resource of ribosomes exists in the simulated cell. In another example, biological facts 70 may be that more than one simulated ribosome can may be attached or allocated to a simulated mRNA strand at a certain time. In another example, biological facts 70 may be that the translation is directional, such that it progresses from the 5′ to the 3′ end of the simulated mRNA strand.

In another example, system 100 may consider the biological fact 70 that a single ribosome may occupy several codons when moving alongside the mRNA molecule (due to its size). Therefore, several ribosomes alongside the same mRNA may be physically forced to keep a minimal distance from each other.

In another example, system 100 may consider biological properties 70 such as initiation rates, which may be affected by properties of the simulated mRNA strands, by properties of initiation factors, and by global factors such as the concentrations of simulated ribosomes and simulated translation factors. In the example of FIG. 2, tᵢₙᵢₜⁱ denotes the average time it takes a ribosome to attach to a specific (index i) simulated mRNA molecule.

In another example, system 100 may consider biological properties 70 such as translation time which may be different for each codon. The translation time may be related to the local biophysical properties of the mRNA strands and their interactions with translation factors and/or the availability of translation factors (e.g., tRNA levels). In the example of FIG. 2, tₜᵣₐₙₛₗₐₜᵢₒₙ^{c}^{ᵢ} denotes the translation time of a specific codon (index c) on the i-th simulated mRNA strand.

In another example, system 100 may consider biological properties 70 such as a simulated ribosome's diffusion time, e.g., the time it takes for each ribosome to become available for translation after completing a translation of an mRNA molecule. In the example of FIG. 2, t_{diffusion} denotes a diffusion time of a ribosome after completing the process of translation, and mᵢ denotes the length of a corresponding i-th mRNA strand.

During development of system 100, a software-based algorithm was used to determine the most time-consuming tasks for modelling the whole-cell process of mRNA translation. Experimental results have shown that the most time-consuming task was the calculation of one or more delay values, referred to herein as a “time-event vector” or “time vector”. In the exemplary implementation of whole-cell mRNA translation simulation, the time event vector was defined as the time that was expected to elapse between association of a ribosome to an mRNA, and the time when that ribosome was ready to decode another mRNA sequence. In other words, the time-event vector of this example was defined a real-world delay, denoted in milliseconds, and consistent of the duration of ribosome initialization, codon translations and ribosome diffusion.

It may be appreciated that the calculation of the time-event vector is highly sequential due to its cumulative nature and the dependency of one ribosome's time-event vector on the time-event vector of another, previous ribosome.

In other words, experiments have shown that a significant bottleneck of a software-based simulation model is the synchronization among client (e.g., mRNA) entities: The simulation could advance only after calculating the state of all mRNA molecules. It may thus be appreciated that hardware-based (e.g., FPGA) implementation of model 100 may accelerate this stage, as FPGA is suitable for accommodating large amounts of synchronized replicas of hardware instances.

It should also be noted that serial, software-based implementation of whole-cell process simulation may produce a bias effect on the global synchronization between sub-processes. For example, a first biological sub-process (e.g., translation of a first mRNA strand having a first length) may affect global synchronization differently (e.g., more adversely) than a second biological sub-process (e.g., translation of a second mRNA strand, having a different length). It may be appreciated that parallel, hardware-based implementation of whole-cell process simulation may inherently separate the two sub-processes, and therefore be devoid this bias effect.

According to some embodiments, system 100 may include, or be implemented by a processor-embedded hardware circuit. For example, embodiments of system 100 have been implemented, and tested on the Xilinx ZCU104 evaluation board, that consists of a Zynq chip containing several CPUs (central-processing-units) cores alongside an FPGA.

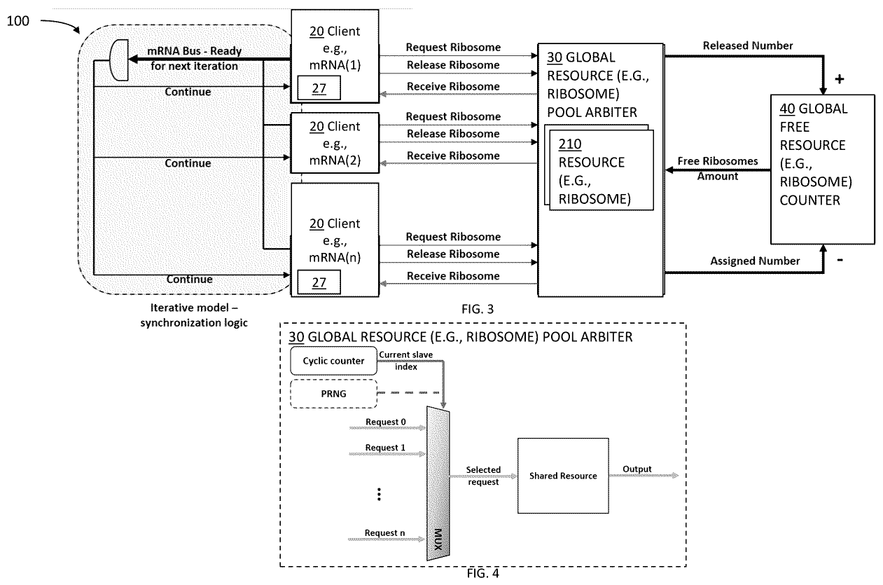

Reference is now made to FIG. 3, which is a high-level block diagram depicting hardware modules of a system 100 for performing whole-cell process simulation, according to embodiments of the present invention.

As depicted in FIG. 3, system 100 may include one or more (e.g., a plurality) of hardware modules, referred to herein as client modules 20 or mRNA modules 20. Each mRNA module 20 may represent a specific mRNA strand entity in the simulated cell, and may contain or store specific codons information that pertains to that mRNA strand.

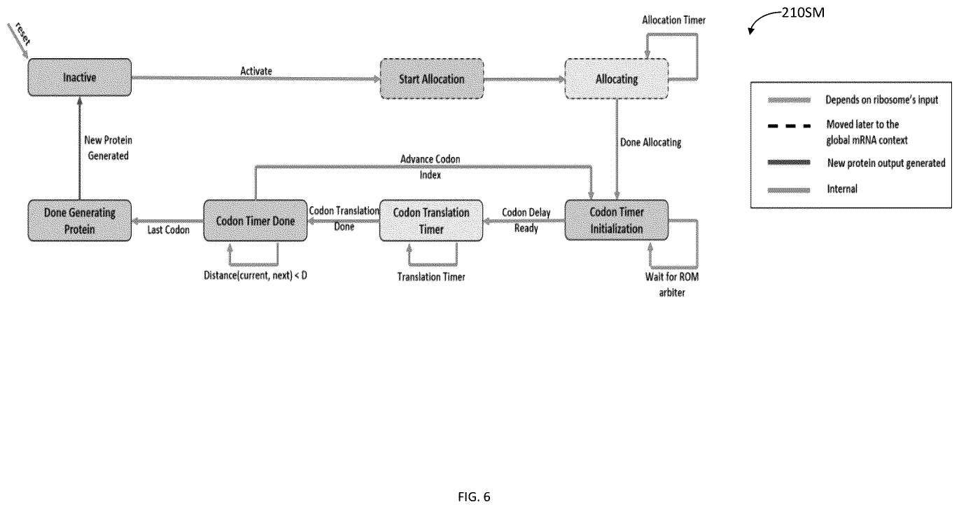

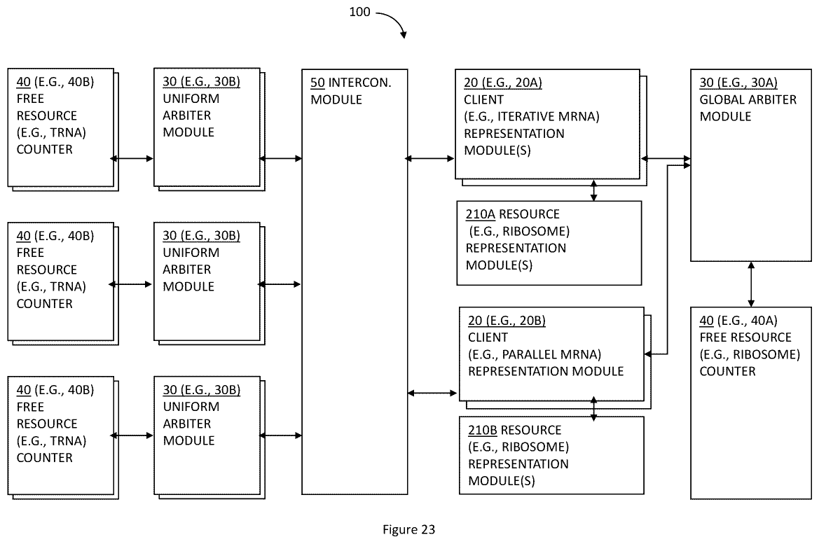

Additionally, system 100 may include a plurality of resource hardware modules 210 also referred to herein as ribosome hardware modules 210, each representing a respective simulated resource entity (e.g., ribosome) of the simulated cell. According to some embodiments, and as depicted in the example of FIG. 3, ribosome hardware modules 210 may be managed globally (e.g., in relation to the entire simulated cell), or locally per each mRNA module 20, as elaborated herein (e.g., in relation to FIG. 23). Ribosome hardware modules 210 may include a state machine 210SM (e.g., as depicted in FIG. 6) which are configured to track states of the ribosomes.

For example, state machine 210SM may have an initial, “Inactive” state, representing a state of a simulated ribosome that is not allocated to a simulated mRNA strand. Once a ribosome is being activated, or allocated by global arbiter 30, state machine 210SM may advance to allocation states, such as “Start Allocation” and “Allocating”, that model the allocation time of a simulated ribosome to a simulated mRNA strand. State machine 210SM may continue to subsequent codon states, such as “Codon Timer Initialization”, “Codon Translation Timer” and “Codon Timer Done”. The codon states model the translation of the codons of the specific mRNA. These states are repeated until the simulated ribosome finishes translating all the codons on the simulated mRNA strand. These states also allow keeping the distance (e.g., a minimal number of simulated codons) from the consecutive simulated ribosome on the simulated mRNA strand, by waiting in the “Codon Timer Done” state until the next ribosome is far enough. State machine 210SM may include a final state, “Done Generating Protein”, in which the resource module 210 representing the simulated ribosome reports generating the relevant protein, and state machine 210SM returns to the initial “Inactive” state.

Additionally, each mRNA module 20 may manage or handle interactions of the simulated mRNA strand with simulated ribosomes, and keep track or count a quantity of simulated, generated proteins. For example, each mRNA module 20 may include a protein counter module 27, configured to count a quantity of simulated proteins that are generated by that mRNA module 20.

As depicted in FIG. 3, system 100 may also include at least one global resource arbiter 30, also denoted herein as ribosome pool arbiter 30. Ribosome pool arbiter 30 may be a hardware module that may be configured to manage assignment of simulated ribosomes to different mRNAs modules 20.

Additionally, system 100 may include a free resource counter 40, also referred to herein as ribosomes counter 40. Ribosomes counter 40 may be a hardware module configured to keep track of the state of each simulated ribosome. This state may include a current codon index, a translation time, and the like. Additionally, ribosomes counter 40 may be configured to collaborate with ribosome pool arbiter 30 to keep track of the overall quantity of ribosomes that are available for translation in the simulated cell.

Additionally, system 100 may include a plurality of timers, associated with, or included in one or more (e.g., each) hardware entity (20, 210, 30, 40) to model allocation, translation, and diffusion delays. In some embodiments, these timers may be decremented in each global clock cycle. The initialization value of those timers was chosen by normalizing all delays from seconds to clock cycles. In E. coli, for instance, each timer decrement models 1 ms in “real” cell time. Therefore, when running the hardware with input clock frequency f, the timers decrement phase of the model can potentially take 1 ms/1/f=0.001*f [Hz] times faster in hardware. Thus, for example for f=100 MHz, the hardware runs 100,000 times faster (in the decrement phase) than a real cell.

As shown in the example of FIG. 3, a first approach, by which one global arbiter 30 may keep track of the available ribosomes may be chosen.

In this example, ribosome arbiter 30 may receive requests from one or more mRNA modules 20 to allocate a ribosome from a pool of available ribosomes, and may subsequently grant requested ribosomes to the requesting mRNA modules 20, when available. Additionally, or alternatively, ribosome arbiter 30 may receive releases signals from one or more mRNA modules 20, and may thereby retrieve simulated ribosomes back to the pool of available ribosomes. Ribosome arbiter 30 may continuously (e.g., repeatedly, over time) updates the free ribosomes' pool, to maintain correct inventory of this simulated resource.

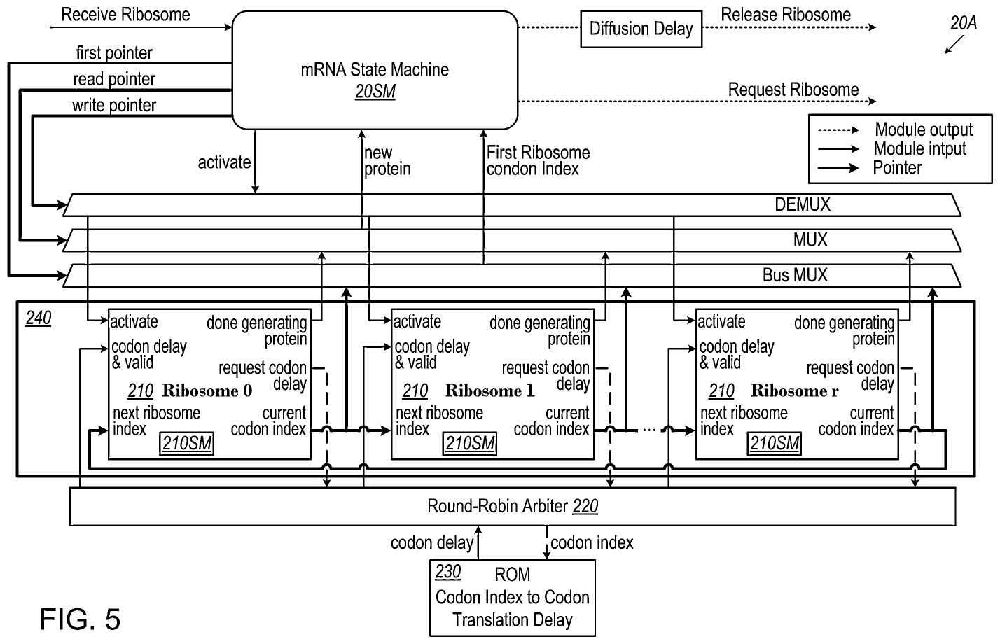

In an alternative approach, management of the pool of simulated resources (e.g., ribosomes) may not be performed globally. For example, a small local buffer of ribosomes, that propagates in a concatenated manner between the mRNA molecules may be used as elaborated herein (e.g., in relation to FIG. 5).

It may be appreciated that arbiter 30 may be required to arbitrate allocation of simulated resource entities (e.g., ribosomes) among a very large number (e.g., thousands) of clients endpoints (e.g., mRNA modules 20), depending on the number of mRNA molecules which system 100 is configured to simulate. Additionally, arbiter 30 may be required to arbitrate allocation of simulated resource entities with uniform probability. In other words, each requesting client 20 (e.g., simulated mRNA strand) should receive the resource (e.g., simulated ribosome) with equal probability, to best represent the environment of a simulated cell.

It should be noted that existing implementations of hardware arbiters are designed for only a few endpoints (i.e., multiple cores accessing the same shared memory), and are not required to be strictly uniform.

Reference is now made to FIG. 4, which is a schematic illustration of an arbiter module 30 which may be included in system 100, according to embodiments of the invention.

During development of system 100, two implementations for the global arbiter 30 were examined: a “round-robin arbiter” and a “uniform arbiter”. The round-robin arbiter is a deterministic arbiter which is commonly used in hardware implementations. It typically consists of a cyclic counter that iterates sequentially over all clients (e.g., mRNA 20 indices). Due to this deterministic counting, the round-robin arbiter does not necessarily satisfy the requirement of uniform probability, and suffers from inherent bias in resource allocation.

The uniform arbiter 30 is a novel variation of the round-robin arbiter 30, in which the inherent bias was fixed. Instead of a deterministic counter, the uniform arbiter 30 includes a hardware efficient uniform pseudo-random number generator—UPRNG. By doing so, the arbiter first randomizes an index and then examines the signals of the indexed mRNA. This arbiter randomizes an index regardless of the state of the corresponding mRNA module 20 (e.g., whether the mRNA 20 is currently requesting a ribosome 210 or not). As shown by the dashed line of FIG. 4, by replacing the cyclic counter with a pseudo-random number generator (PRNG) a uniform arbiter may be obtained.

According to some embodiments, the one or more client (e.g., mRNA) modules 20 may be implemented by either one of the following hardware design approaches: a parallel hardware design approach, an iterative hardware design approach, and any combination thereof.

The parallel hardware design approach would require using as many replicas of processing units as necessary to improve timing performance. This approach typically results in high chip area consumption and high throughput. The iterative hardware design approach may require employing the same processing unit for different workloads when possible. This approach typically results in low chip-area consumption but also with lower throughput.

While the parallel approach may improve the overall performance (e.g., throughput) with respect to software-based implementations of whole-cell simulation, it may not support simulation of as many biological entities (e.g., not facilitate sufficient mRNAs client modules 20) as required for simulation a single FPGA chip.

According to some embodiments, system 100 may include different types or implementations of client (e.g., mRNA) modules 20, that may complement each other in a synergistic manner, as elaborated herein.

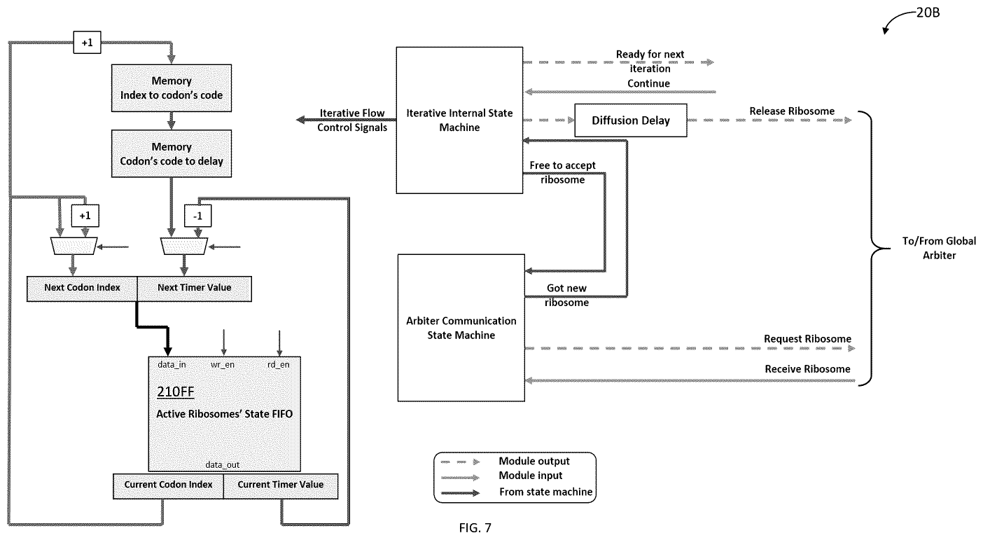

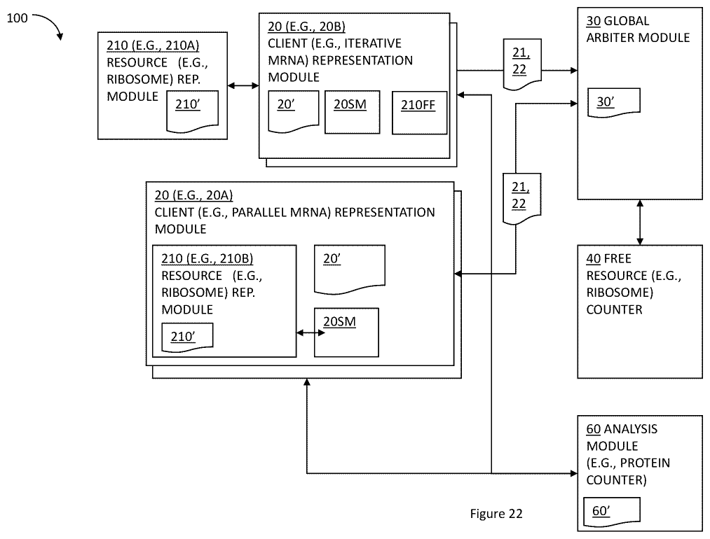

A first client (e.g., mRNA) module 20 type may be referred to herein as a parallel client (e.g., mRNA) module 20, as elaborated herein e.g., in relation to FIG. 5 and FIG. 6. A second client (e.g., mRNA) module 20 type may be referred to herein as an iterative client (e.g., mRNA) module 20, as elaborated herein e.g., in relation to FIG. 7. Both parallel and iterative models have the same high-level block diagram representation, as depicted herein e.g., in FIG. 22 and FIG. 23.

The terms “parallel client 20” and “parallel mRNA 20” may be used herein interchangeably when relating to the non-limiting example where system 100 is used for simulating a biological process of mRNA translation in a simulated biological cell. Similarly, the terms “iterative client 20” and “iterative mRNA 20” may be used herein interchangeably when relating to this non-limiting example.

Reference is now made to FIG. 5 which is a schematic block diagram depicting an exemplary implementation of a parallel client (e.g., mRNA) module 20A, with hardware resource (e.g., ribosome) modules 210 that may be included in, associated to, or allocated to client (e.g., mRNA) module 20A, according to embodiments of the present invention.

As depicted in the example of FIG. 5, parallel mRNA module 20A may provide a hardware representation of a simulated mRNA strand. The behaviour of the simulated client (e.g., mRNA strand) may be implemented in hardware by a dedicated state machine 20SM. Additionally, parallel mRNA module 20A may include resource modules 210, each providing a hardware representation of a corresponding simulated resource (e.g., ribosome) of the simulated cell. The behaviour of each individual simulated resource (e.g., ribosome) may be implemented as an individual state machine).

The mRNA state machine 210SM may be responsible for communicating with global arbiter module 30, to activate, or deactivate each hardware ribosomes module 210, thereby simulating a condition in which the relevant simulated ribosome is actively translating codons of the simulated mRNA strand, or not. According to some embodiments, hardware ribosomes modules 210 of parallel mRNA 20 may, once activated, operate autonomously and in parallel to each other, thereby simulating behaviour of ribosomes in a real biological cell. Additionally, each mRNA module 20 (20A, 20B) may similarly operate autonomously and in parallel.

In the parallel approach, it may be desired to run all client modules (e.g., mRNA 20) and ribosome-related modules (e.g., 30, 40, 210) as autonomously as possible, to simulate operation of respective mRNA strands and ribosomes in a living cell. The major difficulty introduced by this approach is the assignment or allocation of resources (e.g., representing ribosomes) to clients 20 (e.g., representing mRNA molecules).

As shown in FIG. 5, each mRNA module 20 may include, or may be associated with, a concatenated structure 240 of hardware-implemented ribosomes 210. Each mRNA module 20 may have a static allocation of hardware ribosomes 210. The hardware ribosomes 210 may be initiated in an inactive state, and may be activated one by one when mRNA module 20 is assigned, or allocated new ribosomes 210 from arbiter 30.

The concatenated structure 240 of hardware-ribosomes 210 may be, or may include a memory-based First In First Out (FIFO) queue, which consists of read pointer and a write pointer, where the read pointer points to the oldest occupied entry (e.g., the oldest active ribosome), and the write pointer points to the first unoccupied entry that can be used once a new entry is pushed to the FIFO (e.g., the next ribosome to be activated).

Additionally, or alternatively, one or more (e.g., each) mRNA module 20 may include an mRNA state machine 20SM, configured to manage activation, allocation of hardware ribosomes 210 to, and release of hardware ribosomes 210 from that client (e.g., mRNA) module 20. In such embodiments, mRNA state machine 20SM may manage a set of pointers, allowing it to follow up on the status of individual hardware ribosomes 210 in the concatenated structure 240 of hardware-ribosomes 210.

One such pointer of concatenated structure 240 may be a “write” pointer, that may represent an index or identification (e.g., denoted “Ribosome 0”, “Ribosome 1”, . . . , “Ribosome r” in FIG. 5) of the next inactive hardware ribosome 210. When mRNA module 20 (20A) receives, or is allocated a new ribosome from arbiter 30, the hardware resource (e.g., ribosome) module 210 that is pointed to, or identified by the write pointer will be activated.

Another such pointer of concatenated structure 240, referred to herein as a “first” pointer may represent an index of the simulated ribosome that is located at the beginning of the simulated mRNA strand, e.g., closest to the 5′ end of the mRNA strand represented by mRNA module 20. In other words, “first” pointer may represent the last hardware resource (e.g., ribosome) entity 210, that was activated or allocated to client (mRNA) entity 20. Using this pointer, mRNA state machine 20SM may monitor the first ribosome's index until it is far enough along the simulated mRNA strand represented by mRNA module 20 (e.g., such that the first simulated codons are not occupied), thereby enabling mRNA module 20 to accept, or be allocated a new ribosome module 210.

Another such pointer of concatenated structure 240 may be a “read” pointer, which may represent an index of the oldest active resource (ribosome) module 210. In other words, the read pointer may indicate, or point to the simulated ribosome (represented by module 210) that is closest to the 3′ end of the simulated mRNA strand (represented by mRNA module 20 (e.g., 20A)), will be the next to generate a protein, and will subsequently be deactivated.

According to some embodiments, instead of maintaining a local copy of the mRNA delays' table for each hardware ribosome 210, a round-robin arbiter 220 and a single read-only memory (ROM) 230 were used for all hardware ribosomes of the same mRNA module 20 (20A). That is important because the hardware utilization of each resource (ribosome) module 210 has been found to significantly limit the global utilization of hardware (e.g., FPGA) resources of system 100. Round-robin arbiter 220 may iterate over all connected resource (ribosome) modules 210. Per ribosome, round-robin arbiter 220 may associate the codon index (e.g., the index of the currently translated codon) as the address to the codon's in ROM 230 and reports back the content of the ROM 230 to the connected ribosome. For example, round-robin arbiter 220 may utilize ROM 230 as a lookup table, configured to receive an address representing a specific simulated codon or codon type, and fetch a corresponding codon translation delay value.

The diffusion of the ribosomes (i.e., the time it takes a ribosome to be usable again by other mRNAs) may be modeled by delaying the ‘released’ signal of mRNA module 20.

Reference is now made to FIG. 6 which is a schematic block diagram depicting an exemplary implementation of a state machine 210SM that may be included in one or more (e.g., each) ribosome module 210, according to some embodiments of the invention.

According to some embodiments, the dashed-line states depicted in FIG. 6 may be moved to the global mRNA context, as elaborated herein.

Here, the delay that the state machine adds to the ribosome timing (including waiting for the ROM arbiter) is negligible in relation to the timer's delays.

As the hardware-ribosome module is instantiated multiple-times in the hardware, it is important to keep it as compact as possible. Placing the mRNA's ROM in the mRNA module with a common arbiter instead of keeping a copy for each ribosome results in each ribosome consuming 65 lookup-tables (LUTs) and 31 Flip flops (FF) on average. To further improve that, the allocation states (denoted by a dashed line in FIG. 6) was removed from the ribosome's state machine and added allocation logic to the mRNA state machine. That is possible as only one ribosome can be at the allocation phase (at the 5′ end) at a given time. By doing so, it was possible to reduce the size of the hardware-ribosome to 48 LUTs and 22 FFs on average, improving the LUTs and FFs consumption by 26% and 30%, respectively.

Reference is now made to FIG. 7, which is a schematic block diagram depicting an exemplary implementation of an iterative client (e.g., mRNA) module 20B, according to embodiments of the present invention.

In the implementation example depicted in FIG. 7, iterative mRNA 20B may not include individual hardware resource modules 210 that represent simulated resource entities (e.g., ribosomes) of the simulated biological cell. Instead, iterative module mRNA 20B may include a FIFO queue 210FF that stores the current state of the current active simulated ribosomes that are allocated to the simulated mRNA strand, represented by iterative mRNA module 20B. For example, FIFO 210FF of a specific iterative mRNA module 20B may store information pertaining to each simulated allocated resource ribosome, including for example its current index (e.g., a current codon that is being translated), a remaining translation time, and the like.

Iterative mRNA module state machine 20SM may iterate over all its entries in FIFO 210FF to manage the state of the current simulated active ribosomes. Since each mRNA can have different number of ribosomes this technique may require synchronization between the mRNA modules to avoid a condition in which simulated mRNA strands with smaller number of ribosomes, will be translated faster than simulated mRNA strands with more ribosomes (because there are more calculations to be done in each iteration over the FIFO). Therefore, iterative mRNA modules 20B may not be independent of each other and may be synchronized such that they will not advance to the next FIFO iteration before all iterative mRNA modules 20B are done with the current iteration.

As shown in FIG. 7, iterative mRNA module block may include a FIFO that may contain the state of all active ribosomes modules. The state of the ribosome is read and updated from the FIFO by the state machine. When a specific simulated ribosome advances to the next codon, the next translation delay is brought from the concatenated memories shown above. This module may also contain a separate state machine for communication with the global ribosomes' pool arbiter.

To improve the number of supported mRNAs and ribosomes, the iterative approach was then examined. The iterative mRNA module block diagram is shown in FIG. 7. The codons' data may be stored in two concatenated memories—one containing the codon's code and one mapping the code to translation delays. Instead of having multiple replicas of hardware ribosomes (as in the parallel case), only the state of each active ribosome (the current codon index and remaining translation time) inside a cyclic FIFO is kept.

To manage the system, two separate state machines may be introduced. The first is to internally iterate over all active ribosome and advance their state. To synchronize the timing of all mRNA molecules, the state machine outputs a “ready” signal upon iteration completion and waits for all other mRNAs before moving to the next iteration (see the dash-circled area in FIG. 3). The synchronization here affects the performance greatly as the “busiest” mRNA (the one with most active ribosomes) will hold back the update of all the other mRNAs. By doing so, the “busiest” mRNA dictates the time it takes the model to finish each step.

The second state machine is responsible for communication with the global arbiter. This separation is done to have the global arbiter run as freely as possible at the design clock speed. This feature is later shown to compensate for the lower hit probabilities of the uniform-arbiter for the iterative case. Finally, as before, the diffusion time is modeled by delaying the “release” signal.

The different implementations (20A, 20B) of client module 20 provide a tradeoff between speed and resource consumption that may be exploited according to a predefined required target. Experimental results have shown that due to the synchronization overhead of the iterative implementation 20B of client module 20, as explained above, iterative client 20B may run 1-2 orders of magnitude slower than the parallel implementation 20A client module 20. In a complementary manner, iterative mRNA module 20B may not contain hardware instances of resource (e.g., ribosome) modules 210, and may therefore consume less hardware resources than parallel module 20A. Experiments have shown that a single FPGA may accommodate 1-2 orders of magnitude more mRNAs in the iterative model 20B in comparison to the parallel mRNA module 20A.

According to some embodiments, the plurality of client hardware modules 20 in system 100 may include at least one client hardware module of an iterative client module 20B type, and one or more client hardware modules of a parallel client modules 20A type. As elaborated herein, such combination of parallel client modules 20A and iterative client modules 20B may provide a synergic effect. For example, system 100 may predict one or more interaction values 20′ representing an aspect of interaction between simulated client entities (e.g., mRNA strands) and simulated resource entities (e.g., ribosomes) in the simulated cell. In some embodiments, system 100 may be configured to use the at least one client hardware module 20 of the iterative client module 20B type as initial screening, to identify a subset of simulated client entities in the simulated biological cell, characterized by a first desired objective. System 100 may be configured to subsequently use the one or more client hardware modules of the parallel client modules 20A type, to select a simulated client entity in the simulated biological cell, that is characterized by a predefined synthetic biological objective.

For example, system 100 may be employed to determine an optimal mutation of a specific synthetic biological objective, e.g., a protein. In such cases, system 100 may initially be applied to a large number of mRNA mutations by using the iterative model 20B to identify a subset of the plurality of mRNA mutations. The subset of mutations may be characterized by a first desired objective (e.g., a protein that perform a desired function). System 100 may subsequently be applied to the subset of mRNA mutations by using the parallel model 20A to focus on the most interesting mRNA mutations that provide a second desired objective (e.g., produce the desired protein in the most time-efficient manner). It may be appreciated that such combination of client module architectures may facilitate (i) examination of more mutations than would be possible using the parallel implementation 20A alone, and (ii) examination of these mutations 1-2 orders of magnitude faster than would be possible using the iterative module 20B alone.

A further demonstration of the effectiveness of hardware modelling, is presented by running the FGM and BGM algorithms presented in Zur et al., “Algorithms for ribosome traffic engineering and their potential in improving host cells' titer and growth rate”, Sci. Rep. 10, 21202 (2020), herein incorporated by reference in its entirety, using the iterative hardware model and are described below. In that paper, optimization algorithms for improving the allocation of ribosomes in the cells by decreasing their traffic jams during translation were introduced. The algorithms introduced silent mutations within the coding regions. These do not affect the linear chain of the encoded protein amino acids but can affect the cell growth rate. Specifically, the algorithm introduced there are:
(1) Forward Gene Minimization (FGM): incorporates all silent mutations (from the beginning of the ORF) that improve the free ribosomal pool while not reducing/increasing the mRNA's translation rate beyond some threshold. In each iteration, the mRNA that increases the free ribosomal pool the most is selected; and
(2) Backward Gene Minimization (BGM): Similar to FGM, while starting at the end of the modified region in the ORF and traverse backwards.

To reduce communication overhead as much as possible, we used the ARM cores in the Zynq processor to operate the FPGA and run the optimization algorithms.

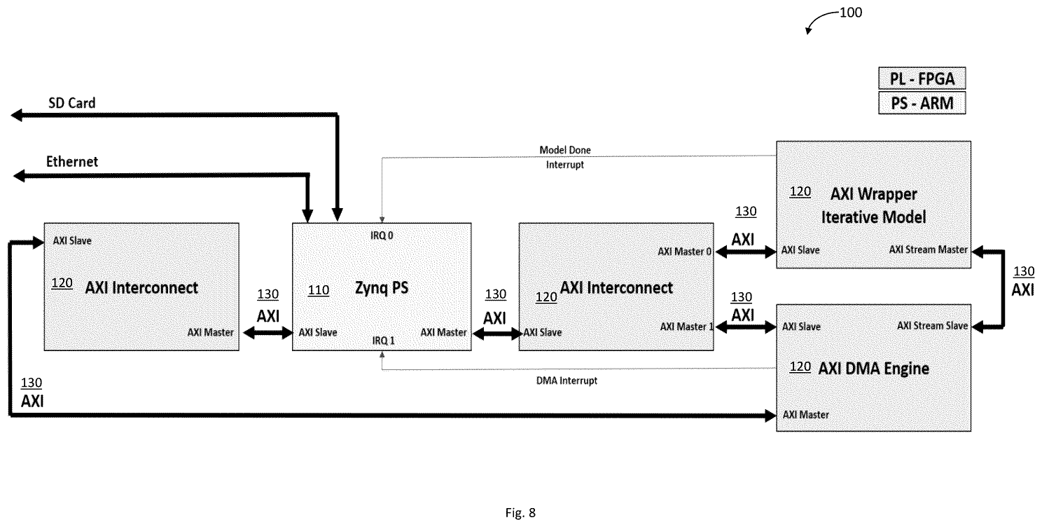

FIG. 8C is a schematic block diagram depicting implementation of a system for simulating whole-cell biological processes, including the FPGA's programmable logic (PL) part (in light blue) and a processing system (PS) part implemented by one or more processors (light orange), according to embodiments of the present invention. System 100 of FIG. 8 may be, or may include the same system 100 as that of FIG. 3.

As elaborated herein (e.g., in relation to FIG. 22), the one or more client hardware modules 20, resource hardware modules 210, arbitration hardware modules (arbiter module 30) and resource counter 40 may be included, or may be implemented, at least in part as programmable logic (PL) 120 in a dedicated hardware electrical circuit such as an FPGA or ASIC chip. Additionally, system 100 may include one or more processors 110 (also referred to herein as CPUs, processing system (PS), CPU cores, processing cores, ARM or Zynq), embedded (as commonly referred to in the art) in the dedicated hardware electrical circuit (e.g., FPGA or ASIC chip). The one or more processors 110 may be configured to communicate with PL 120 to employ hardware modules (e.g., 20, 30, 40, 210) of PL 120 to simulate whole-cell biological processes.

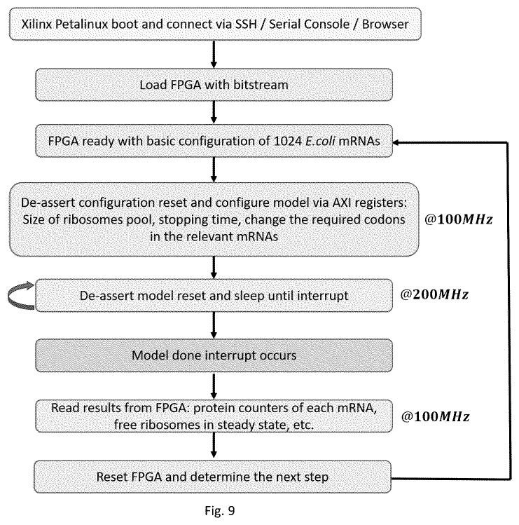

Reference is also made to FIG. 9 which is a schematic flow graph illustrating an order in which operations may be carried out by system 100 for simulating whole-cell biological processes, according to embodiments of the present invention.

As shown in FIG. 8, to connect the PL 120 (also referred to herein as FPGA or FPGA model) to the CPU cores 120, system 100 may use one or more on-chip dedicated interfaces 130, between the cores 110 and the FPGA 120. referred to herein as AXI 130, Interface 130 may be implemented by an on-chip communication bus such as the Advanced extensible Interface (AXI), and may be also referred to herein as AXI 130.

AXI interface 130 may generate a memory-mapped register read-write interface to the hardware. Also, for reading large amounts of data from the FPGA 120 (for example—read all protein counters 27 from all mRNA modules 20), a direct-memory-access (DMA) engine can be connected to allow the FPGA 120 direct access to the on-board CPU 110 memory. When the FPGA model 120 reaches the configured stop time, it may raise an interrupt to the dedicated interrupt pins of the ARM cores 110.

Embodiments of the present invention provide a novel approach that can be very useful in synthetic biology. Whole cell modelling and engineering may be performed based on the dedicated hardware of system 100.

In an exemplary implementation of system 100, it has been experimentally observed that the iterative model runs up to 260 times faster than equivalent software models. For example, a single optimization procedure took 30 hours for the hardware iterative model, and was approximated to take more than 8 months with the equivalent software model.

In comparison with the parallel model, the iterative model was found to perform slower, but was also capable of accommodating more mRNA modules 20 (e.g., up to 1024 mRNAs) and ribosomes modules 210.

The parallel model was found to work much faster (up to 4690 times faster) than equivalent software models. However, the parallel model was found to accommodate less simulated molecules (e.g., up to 128 mRNAs and 4096 ribosomes) in relation to the iterative model.

These results could be anticipated since this is a common tradeoff in designing hardware—runtime vs. chip area. In the parallel model, the ribosomes can run in parallel to each other for the price of having them implemented in hardware. Oppositely, in the iterative model, the ribosomes are being run sequentially (and therefore, slower) by the mRNA state machine and therefore can only consume the area needed for their state.

As the iterative model can accommodate more mRNA molecules, it suits best for a whole cell modelling and optimization. In order to explore a smaller part of the cell, the parallel model can be used to cover much more configurations in the same amount of time. Accordingly, it is possible to first run the FGM and BGM algorithms on large amounts of mRNAs and then, for example, to use the simulated annealing algorithm to further optimize the 64 “best” mRNA molecules using the parallel model.

Embodiments of the present invention can be used for modelling other types of intracellular competitions and for changing intracellular conditions. The description of embodiments of the invention herein above demonstrates modelling competition over a limited ribosomal pool as currently this is the most studied intracellular model in the field mainly due to the fact that most of its parameters can accurately be estimated from experimental data. It is important to emphasis the fact that due to the competition on limited cellular resources such as ribosomes and tRNAs, even a small intracellular circuit (e.g., 1-3 genes) can affect the entire cell and should be engineered based on a whole cell model. This is specifically true when the expression levels of the circuit need to be high and induce huge load on the host. It should be emphasized that translation consumes more than 75% of the energy in the cell; thus, it is not surprising that translation is an important aspect in such cases.

However, it would be apparent to those skilled in the art, that similar approach can be used for modelling other intracellular aspects such as competition of tRNA, miRNA, transcription factors, and more, alongside more details related to the biophysical process (e.g., operon structure and re-initiation). For example, for modelling competition of tRNAs, examining a similar approach to the arbitration over the finite ribosomal pool is suggested. For example, 61 pools of tRNA molecules (excluding the stop codons) that receive requests from all ribosomes may be considered. In that embodiment the challenge may be the routing of all requests from all ribosomes to this pool in a ribosomal saturated cell.

It is important to emphasize that during the intracellular engineering process, the parameters of the models may change. For example, the concentrations of the tRNA molecules mainly impact the codons' translation delay and the values used here are the average based on measurements from real cells and therefore already include the influences of various tRNA concentrations. Thus, the demand for tRNA molecules in the cell might change when inducing silent mutations to several mRNAs as suggested in embodiments of the present invention. If the change in the demand is substantial, it might impact the translation delay of several codons. By going back to the first stage shown in FIG. 1 with new experimental data, those variations can be corrected.

Finally, it should be emphasized that the fact that according to experience whole cell models based on differential equation are also very slow; thus, though the resolution of the model usually decreases, this is not a solution to the challenge of performing very fast simulations. This suggests that Hardware solution may also be relevant for accelerating whole cell models based on differential equations.

Moreover, as the main limitation of the parallel model is the amount of hardware ribosomes, a design that efficiently distributes the hardware-ribosomes between the mRNA modules poses a challenge (see the Methods section for more details). A more dynamic approach so the hardware ribosomes could be shared by several mRNAs. This problem, of dynamically allocating a common resource, resembles the way virtual memory is implemented in hardware. A similar approach can be implemented here.

Also, we can consider a solution in which multiple FPGAs are connected to form a large system. Platforms that support several FPGAs already exist in the market today. By using such platforms, we can distribute more mRNAs between the FPGA chips and split the global ribosomes' pool into several small pools that are communicating with each other. In addition, each FPGA can simulate one aspect or stage of gene expression (e.g., one FPGA for transcription, one for transport, one for translation, etc.).

And finally, one can consider implementing an application-specific-integrated-circuit (ASIC). FPGAs are quite comfortable for prototyping as done here, but are quite inefficient in the matter of power consumption, area utilization and operating frequency in comparison to ASICs. Specifically, for example, we can expect that for a full system design, the equivalent ASIC area will be up to 10 times smaller than the FPGA area. Therefore, we can expect that by having an ASIC of the same die size as the FPGA, we can potentially support up to 10,240 iterative mRNAs and 1280 parallel mRNAs. Using the architectures presented in this paper, it is possible to implement a high speed, highly configurable ASIC that can accommodate large numbers of mRNAs and ribosomes.

Also, the light-weight randomization mechanism presented here can be easily adapted to randomize the translation delay of the codons. By doing so, the model can become completely stochastic at the cost of consuming more FPGA resources.

Another feature that can be considered is modelling the degradation of ribosomes and mRNA molecules in the cell. The current architecture of the hardware supports modifying the ribosomes' levels to simulate degradation as the number of ribosomes is a software-controlled parameter of the hardware that can be dynamically changed throughout the simulation. Regarding the mRNA molecules' degradation, to support the degradation feature, the enable signals (that already exist in hardware) for the hardware-mRNA modules, should be routed to the software interface. This is a simple hardware change that can allow the software algorithm to impact the mRNAs' degradation by randomly disabling mRNA molecules according to the wanted heuristic. However, one challenge related to this aspect is related to the lack in the experimental measurements of half-lives of mRNAs and ribosomes.

The parameters of the model (initiation rates, elongation rates, mRNAs codons list with various length and the total number of ribosomes) were based on Levin, D. & Tuller, T. Whole cell biophysical modelling of codon-tRNA competition reveals novel insights related to translation dynamics. PLoS Comput. Biol. 16, e1008038 (2020), herein incorporated by reference in its entirety, and are inferred from ribo-seq experiments. The parameters there were inferred by fitting the biophysical model to ribo-seq data of all mRNAs of E. coli. The number of mRNAs in S. cerevisiae and in E. coli is known in the art, as are the number of ribosomes in S. cerevisiae and E. coli. D (ribosome's size) for E. coli and for S. cerevisiae are also known. The model does not directly consider operon structures in the case of E. coli. This structure specifically affects the initiation rate to coding regions inside the transcript as it is a combination of “direct” initiation and re-initiation (after translation termination or the previous coding region).

However, the right initiation rate to each coding region is not modeled since it was inferred by fitting the biophysical model to ribo-seq data of all the mRNAs of E. coli which reflect both components (direct initiation and re-initiation).

In the case of the comparison of the translation rates from models of the invention and PA, since they are limited to 1024 mRNA molecules in the current FPGA, in each organism, the genes that are at the highest levels in the cell were chosen and each gene type was replicated with proportion to the cellular mRNA level of that specific gene.

The Global Ribosomes' Pool Arbiter Local Release Counter. As previously mentioned, the round-robin arbiter iterates over all mRNAs and therefore, it takes exactly m clock cycles to return to the same mRNA molecule. During that time, ribosome release events may occur. To take that into consideration, we added a release counter for each mRNA release signal. The size of this counter can be determined as follows: given the minimal codon's delay as dₘᵢₙᵢₘₐₗ and D as before, it follows that the maximal number of release events during the arbiter's iteration is given by:

$$⌈ \frac{m}{D * {d}_{min imal}} ⌉ {{︷}_{=}}^{E . Coli , 512 mRNAs} 1$$

For the uniform arbiter, the number of release events that can occur until the arbiter reaches the same mRNA is different. First, the number of clock cycles that the arbiter takes to reach the same mRNA molecule is calculated. Here, this number is not a constant (as in the round-robin case) but a random variable. As shown, the arbiter randomizes an index from 0 to m with probability close to 1/m as it is designed to be uniform.

Thus, we have a list of independent identical distributed (i.i.d) Bernoulli experiments with probability 1/m and we ask what number of experiments is required to receive two success events with high probability. Having E experiments

$$\{ {e}_{i} ∼ B e r n ( \frac{1}{m} ) \} ,$$

we get:

$$p ( {\sum }_{i = 1}^{E} {e}_{i} \geq 2 ) = 1 - p ( {\sum }_{i = 1}^{E} {e}_{i} < 2 ) = 1 - {( \frac{m - 1}{m} )}^{E} - ( \begin{matrix}E \\ 1\end{matrix} ) {( \frac{m - 1}{m} )}^{E - 1} ( \frac{1}{m} ) = 1 - \frac{{( m - 1 )}^{E - 1}}{{m}^{E}} ( m + E - 1 )$$

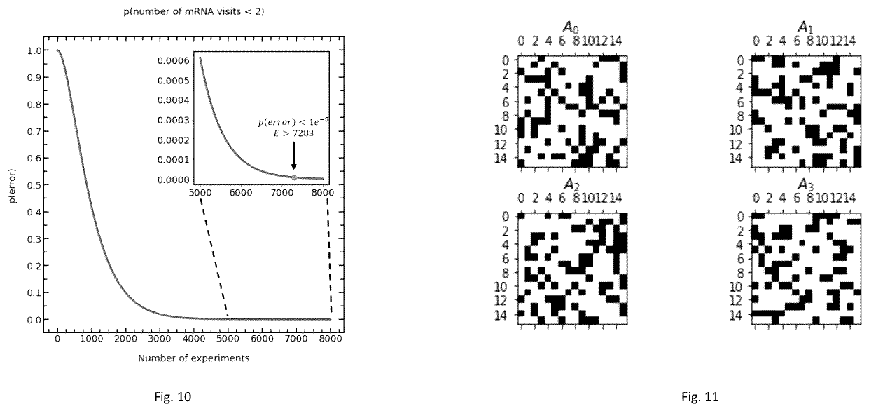

Reference is made to FIG. 10, which is a graph depicting the number of consecutive Bernoulli (with p=1/512) experiments needed for at least two success events with miss probability lower than 1e-5, according to embodiments of the present invention.

As shown in FIG. 9A, for 512 mRNAs, we get:

$$p ( {\sum }_{i = 1}^{E} {e}_{i} \geq 2 ) = 1 - {( \frac{511}{512} )}^{E} - {E ( \frac{511}{512} )}^{E - 1} ( \frac{1}{512} )$$

So, now the maximal number of release events during the arbiter's iteration is with overflow probability Pₑᵣᵣₒᵣ:

$$⌊ \frac{E {\vert}_{p ( {\sum }_{i = 1}^{E} {e}_{i} \geq 2 ) < {p}_{e r r o r}}}{D * {d}_{min imal}} ⌋ {\vert}_{E . Coli , 512 mRNAs , {p}_{e r r o r} = 1 {e}^{- 5}} = ⌊ \frac{7283}{9 * 83} ⌋ = 9$$

Which requires a 4 bits counter for each mRNA molecule.

Uniform-random-number-generators (URNG). There exist plenty of hardware URNGs implementations some of which are optimized specifically for FPGAs. Since we wish to keep the URNG logic as compact as possible, the URNG is the most relevant implementation for our need. There are two types of URNGs: true-URNG and pseudo random URNG (PRNG). The true-URNG is more relevant for cryptographic usages where high quality of unpredictable random sequences should often be generated. The true-URNGs are often based on physical randomness that is generated through various methods and are rather complex. However, for our usage, PRNGs are sufficient.

By following the method suggested in High Quality Uniform Random Number Generation Using LUT Optimised State-transition Matrices|SpringerLink. We had to come up with two matrices that operate on a state register to generate the random number:

$$\{ \begin{matrix}{x}_{i + 1} = A {x}_{i} \\ {o}_{i} = B {x}_{i}\end{matrix}$$

Where xᵢ is the internal state register (with dimension n), oᵢ is the output of the PRNG in the i'th clock cycle (with dimension m), A∈ [symbol] ^{n×n} and B∈ [symbol] ^{m×n} are generation matrices. By choosing the right A∈ [symbol] ^{n×n} matrix, the sequence {xᵢ} can have a cycle of 2ⁿ−1. In Ultrascale+chips, each LUT has 6 entries. Therefore, for having an LUT efficient matrix multiplication of Axᵢ we may keep the number of 1's in A∈ [symbol] ^{n×n} rows below 6. We also wish to make sure that all bits in the state register take place in the calculation of the next state. Software-efficient algorithms for generating adequate matrices as n grows were considered. For our case, small matrices of up to n=32 suffice and for their generation we used the following simplified algorithm:

```
while True:
(1) Aₙₓₙ = 0ₙₓₙ
(2) for i from 0 to n:
  a. k = randomize number of LUT inputs from 4 to 6
  b. indices = randomly choose k indices from 0 to k−1
  c. A[indices] = 1
(3) A_sum = sum A rows
(4) If 0 in A_sum (make sure all state bits effect the next state):
  a. Continue
(5) Calculate xᵢ₊₁ = Axᵢ for i from 0 to 2ⁿ − 1
(6) If |set({xᵢ})| < 2ⁿ − 1 (check if A generates a full cycle):
  a. Continue
(7) Return A
```

Reference is also made to FIG. 11, which presents 16 bit matrices for generating random numbers in hardware, according to embodiments of the present invention. For illustration, for n=16 we get the matrices shown in FIG. 11. It is easy to see that each row or column contains at most 6 ones.

The same approach was used for B∈ [symbol] ^{m×n}. By using this approach, we were able to generate two A_{16×16} matrices (to operate on a 32-bit state register) and one B_{9×32} matrix that produce a random sequence with p value=0.992. For reference, we get p value=0.8 for random sequence of the same length generated by the ‘random’ package in Python. The main reason for this improvement is that Python (for instance) randomizes large integers while the approach here is tuned for small integers.

Apart from generating high quality uniform stream, this PRNG is quite efficient: {A₀, A₁}*xᵢ requires 32 LUTs and B_{9×32}xᵢ requires only 9 LUTs. As the state register here is advanced separately as two concatenated state registers (one for each matrix) when the first state is advanced only after 2ⁿ−1 steps of the second state, an extra 16 bits counter is required.

Parallel model—hardware-ribosomes' buffer size. The maximal theoretical number of active ribosomes operating on the same i-th mRNA of length Lᵢ simultaneously is given by

$$⌊ \frac{{L}_{i}}{D} ⌋ .$$

Therefore, the average number of hardware ribosomes needed for m mRNA molecules is given by:

$$mE [ ⌊ \frac{{L}_{i}}{D} ⌋ ] {\overbrace{=}}^{E . coli} 35 m .$$

By implementing that design, we were able to fit into the FPGA 128 mRNAs at 200 MHz. Here, as opposed to the iterative model, the bottleneck is the LUTs utilization and (not the BRAMs utilization) as the ribosomes occupy most of the FPGA and are composed of the state machine shown in FIG. 6.

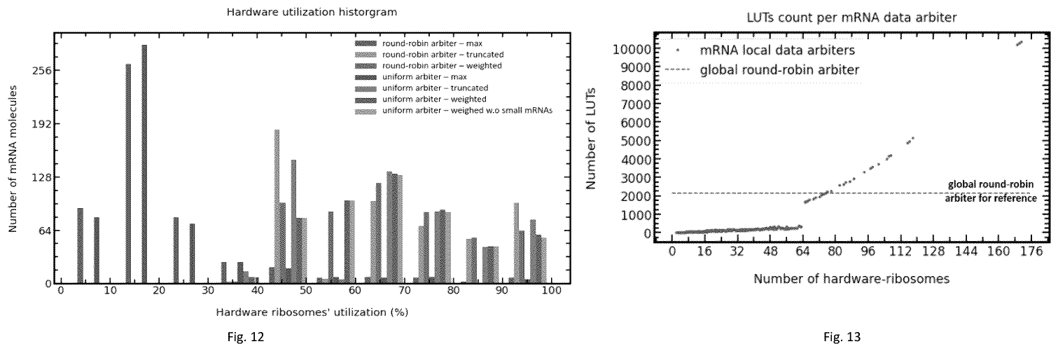

Reference is also made to FIG. 12, which is a graph depicting utilization of allocated hardware ribosomes using different arbiters and different allocation methods, according to embodiments of the present invention. max—each mRNA has

$$\frac{{l}_{i}}{D}$$

hardware ribosomes, truncated—each mRNA has

$$min ( \frac{{l}_{i}}{D} , \frac{total hardware ribosomes}{num mRNAs} )$$

ribosomes, weighted—the total amount of hardware ribosomes is distributed by a weight function.

In real E. coli cells, there are approximately between 20,000 to 50,000 ribosomes and 4100 mRNA molecules. Keeping that ratio when modelling 512 mRNA molecules with 2048 ribosomes, we get the ribosome-hardware utilization histogram shown in FIG. 12. The ribosome-hardware utilization is the maximal number of simultaneously active ribosomes out of the available hardware-ribosomes for the specific mRNA. That shows that the hardware-ribosomes, which consume the most FPGA resources are barely utilized. That is not surprising since the number of ribosomes in a cell is much lower than

$${\sum }_{i = 1}^{m} ⌊ \frac{{L}_{i}}{D} ⌋ .$$

Reference is also made to FIG. 13, which is a graph depicting local mRNA's data arbiter size in LUTs as a function of number of hardware ribosomes, the number of mRNAs that are free to receive new ribosomes as a function of time, according to embodiments of the present invention.

As shown in FIG. 13, as the mRNA has more hardware-ribosomes, its data arbiter consumes more LUTs. That is due to the wide multiplexer in the arbiter that chooses the codon index that is used for the mRNA ROM input (as shown in FIG. 4).

Keeping that in mind, we next investigated a different method to distribute the hardware-ribosomes between the mRNA modules. By summing the maximal number of active ribosomes for all 512 mRNAs in a cell with 2048 ribosomes, the number of hardware ribosomes will be:

$${\sum }_{i = 1}^{m} {mRNA}_{i} max ( active ribosomes ) \approx 2900$$

That means that if we could predict that number, only 2900 hardware ribosomes would be needed for an accurate 512 mRNAs operating with 2048 ribosomes' cell. As mentioned, our hardware can accommodate 4096 hardware ribosomes. For 512 mRNAs and 2048 cell ribosomes, the maximal number of active ribosomes simultaneously is 2048. The question is how to distribute the 4096 available hardware ribosomes between the 512 mRNAs in such a way that each mRNA, even at its most occupied moment, does not miss any ribosomes granted by the arbiter.

We first tried the following approach: assuming 2048 ribosomes are distributed uniformly among 512 mRNAs, the average amount of active ribosomes on each mRNA should be around 4. So, if we double that in hardware-ribosomes per mRNA (as we could fit 4096 hardware ribosomes in a single chip), each mRNA, with high probability, will not saturate its hardware-ribosomes.

As shown in FIG. 12, that leads to 96 mRNAs that saturates their hardware ribosomes for the round-robin arbiter (80 for the uniform arbiter). To further improve that, we seek to find a weight function that can predict the utilization of hardware ribosomes. The factors that should be taken into consideration are:
(1) mRNA length—as it is longer, it is more probable to have more active ribosomes.
(2) Codons translation time—as it takes longer for a ribosome to move along the mRNA, it is more probable to have more active ribosomes.
(3) Entry time—as the initiation delay plus the time it takes a ribosome to clear the mRNA 5′ end, by moving D codons, is longer, the mRNA is expected to request ribosomes at a lower rate.

Taking those parameters into account, we assigned each mRNA the following weight:

$${w}_{i} = log ( \frac{{L}_{i} * {\sum }_{c = D}^{c = {L}_{i}} codon delay}{i n i {t}_{i} + {\sum }_{c = 1}^{c = D - 1} codon delay} )$$

Where initᵢ is the initialization time of the i'th mRNA. Here, we used the logarithm to smoothen the weights. By assigning those weights, with available HR hardware-ribosomes (4096 in Ultrascale+ case), the buffer size of the i-th mRNA is given by:

$${r}_{i} = \frac{{w}_{i}}{\sum {w}_{i}} H R$$

By applying this approach, we were able to reduce the number of mRNAs that saturate their hardware-ribosomes to 57 for the uniform arbiter (see FIG. 12). See the Discussion section for ideas for improving that even further.

Finally, consider the case in which we wish to synthesize a single mRNA molecule and inject it into an existing cell (such as E. coli). In that case, we might wish to model intra-cellular interactions of thousands of different variants of that molecule whilst the rest of the cell remains the same. That use case is highly relevant, for instance, in the process of vaccination development. As the software simulation can run for months, enumerating over thousands of variants, the hardware model becomes quite attractive. In that case, a long simulation can first reveal the actual utilization of each mRNA molecule in the cell. Then, the utilization can be translated to the allocation number of hardware-ribosomes for the cell mRNAs. By doing so, we will be able to fit even more mRNA molecules without hardware saturation in a single FPGA since as shown above, for 512 mRNAs and 2048 ribosomes in the cell we only need 2900 hardware ribosomes (and we can fit 4096).

Improving memory usage of the iterative model. To store the codon's delay list for each mRNA molecule we first used a map between the codon's index to its delay for simplicity. The utilization report revealed that on average, each mRNA module uses one BRAM for the codons' delay ROM. We found that we can reduce its size by replacing it with two concatenated ROM memories as follows. The first ROM maps each codon index to the codon's code. Each codon consists of three nucleotides and each nucleotide can contain one of four possibilities. For coding the nucleotides, only two bits are required (four possibilities). Therefore, each codon can be coded using 6 bits. The second concatenated ROM maps a codon's code to the codon's average translation delay (for the deterministic model the average suffices).

By using the original, single-ROM method, we get:

$${\vert {ROM}_{i} \vert}_{single ROM method} = {2}^{⌈ l {og}_{2} {L}_{i} ⌉} * NDbits$$

Where NDbits denotes the maximal width in bits of the codons' delay (15 bits for E. coli) and Lᵢ denotes the length of the i'th mRNA molecule. We use 2^{┌log}^{₂}^{L}^{ᵢ}^{┐} instead of Lᵢ since, in case of BRAMs, one cannot use a fraction of BRAM that is not a power of 2. By using the double-ROM method, we get:

$${\vert {ROM}_{i} \vert}_{double ROM method} = {2}^{⌈ lo {g}_{2} {L}_{i} ⌉} * 6 bits + 64 * NDbits$$

The term “NDbits” denotes the number of bits required for representing a delay value in the module. In E. coli, only 15 bits may be required. For the codon code to codon delay map, we need NDbits LUTs (6-inputs LUT). In other words, the codon code to codon delay table can be implemented as NDbits 6-input LUTs (15 LUTs in E. coli), where LUT may be implemented as a memory element (e.g., ROM) having 6 bits address and one output bit. That is important as the bottleneck here is BRAMs and not LUTs. Therefore, the average improvement by implementing the double-ROM method is given by:

$${E [ \frac{{\vert ROM \vert}_{double ROM method}}{{\vert ROM \vert}_{single ROM method}} ]}^{- 1} = {( \frac{6}{NDbits} + {\sum }_{i} \frac{64 * p ( {mRNA}_{i} )}{{2}^{⌈ l {og}_{2} {L}_{i} ⌉}} )}^{- 1} {\overbrace{=}}^{E . Coli} 1.85$$

According to some embodiments, client (e.g., mRNA) module 20 was implemented as a parametric RTL module with its own: state machine, ribosomal state management, initiation time, diffusion time and codon's delays list, as follows:

To model allocation, translation, and diffusion delays, we use timers which are decremented each clock cycle. The initialization value of those timers is chosen by normalizing all delays from seconds to clock cycles. In E.Coli, for instance, each timer decrement models 1 ms in “real” cell time. Therefore, when running the hardware with input clock of frequency f(MHz), we get that the timers' decrement phase of the model can potentially take 1 ms/(1/f)=1 ms*f seconds. For f=100 MHz we get that the hardware runs 100,000 times faster (in the decrement phase) than a real cell. Next, the state of the ribosomes is stored in two SRAM memories. Each ribosome is assigned a unique index/address. The first SRAM maps each ribosome to the current codon index along the mRNA molecule. The second SRAM maps each ribosome to the remaining delay time it should wait before advancing to the next codon. Notice that the allocation and diffusion delays are handled outside the SRAM complex to reduce hardware costs (only one active ribosome can be in the allocation/release state in each time point). For the initialization of the ribosomes' translation timers, a ROM that maps codon index to timer value was used. That is, when a ribosome advances to the next codon, the state machine should retrieve the next timer initialization from that ROM.

The mRNA state machine 20SM constantly iterates over all its active ribosomes and decides the next step. The state machine is also responsible for incrementing the local generated proteins' counter 27 upon ribosome release. And finally, to form a multiple-mRNAs model, all mRNAs are cyclically concatenated as follows: each mRNA has a local free ribosomes FIFO which is written by the previous mRNA (upon ribosome release) and read by the current mRNA and the next one. By initializing the input FIFO for the entire complex, we control the assigned global number of ribosomes of the model. By implemented this design, we were able to fit, into the FPGA, 256 E. Coli mRNA molecules with the maximal theoretical storage needed for their ribosomes' states. By doing so, we reached 90% BRAM utilization and 25% LUT utilization with 70 MHz input clock. We then continued by analysis of the utilization report of the design then continued to further improve the utilization in order to be able to fit more mRNA molecules into the design.

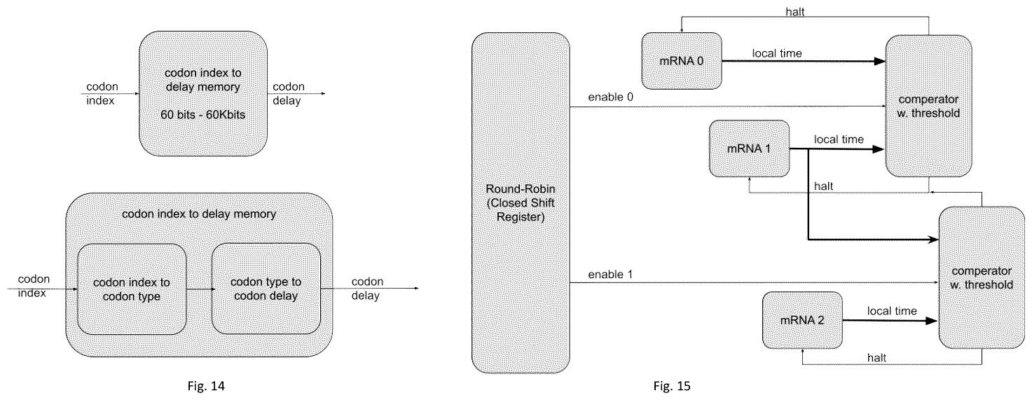

Reference is now made to FIG. 14, which is a schematic diagram showing storage of simulated codons' data, according to embodiments of the present invention. The upper portion table in FIG. 14 depicts a first approach, where a single table may map the codon index to the codon delay value. The lower portion depicts an alternative approach, in which two concatenated tables perform this mapping, where the joint size of these two tables may be smaller than that of the table in the upper portion.

According to some embodiments, mRNA codons' ROM 230 may consist of the delay the simulator should wait for each codon in each mRNA. Due to the fact the translation time of a given codon is independent of the mRNA molecule and only depends on the type of that codon—one can considerably reduce the memory needed for large mRNA molecules. That can be done using two cascaded smaller memories. One that contains the list of the types of the codons in each mRNA molecule and another that contains the translation between codon type to translation time.

mRNA state machine bias: It is easy to see that as in this model, the mRNA state machine iterates over all active ribosomes, the mRNA state machine latency depends on the number of its active ribosomes. That latency is not taken into consideration and causes more occupied mRNAs to generate proteins at a lower rate in the model although that may not be the case in real cells. First, let us examine the influence of the state machine delay via simulation. Using the mRNA iterative module 20B, it has been experimentally observed that a single timer decrement of a codon takes approximately:

$$40 r + 20 ns$$

When r denotes the current number of active ribosomes on the mRNA molecule. The 20 ns constant comes from the state machine delay that is common to all ribosomes. That shows that the state machine delay is not neglectable at all and causes a huge bias to the entire system. In fact, that dependency is linear, meaning that, the time between two consecutive decrements of the same ribosome (Δt_{decrement}) is proportional to the current number of the mRNA's active ribosomes (r):

$$Δ {t}_{decrement} = c * r + const$$

By analyzing the simulation of our hardware model, we had Δt_{decrement}=4*r+2 clock cycles. That demonstrates that the current design's state machine delay may not be negligible in comparison to the codon translation delay. This delay also causes a distortion in the model's timing—ribosomes will be released in hardware long after they are released in “real” cell when they are operating on mRNAs with lots of other active ribosomes. Also, that introduces another issue—causality—the entire simulator is not casual—the “current time” of each mRNA is different. When requesting a ribosome regardless of the current time, the mRNA can receive a ribosome that has not yet been released in “real time”.

mRNA synchronization—one can consider synchronizing the state of all active mRNA molecules in a manner that whilst a given molecule has not done iterating over all its ribosomes, the other mRNA does not advance. Intuitively, this approach is inefficient because it causes plenty of hardware idle times. Moreover, the idle periods are more frequent when we model more mRNA molecules. Although the method of mRNA synchronization might impact the performance of the simulator, it helps with the causality issue—when the state of all mRNAs is synchronized—the system is causal.

Adding current time to the mRNA state—a time counter can be added to the state of each mRNA molecule. The counter should contain the dT passed from the beginning of the simulation. The counter should be advanced only upon completion of the state machine's iteration. In this way, it will be possible to divide the generated protein's counter by the dT of that mRNA molecule to receive the actual generation rate. Those changes were taken into consideration in the later versions of the iterative model presented in the article.

The following approaches were also considered: Halt and advance the slowest—occasionally, when the dT of the current time is large enough between mRNA molecules in the simulator—halt the fastest mRNAs and advance the slowest. Regardless of the implementation in hardware (which might be extremely complicated when having multiple mRNA molecules), it might not solve the problem—longer mRNA molecules are more likely to have more ribosomes and therefore might be consistently slower than all other mRNAs. That might cause frequent halts.

Reference is now made to FIG. 15, which is a block diagram depicting an example for implementation of a synchronization mechanism, according to some embodiments of the invention.

Match time in pairs—As shown in the example of FIG. 15, A cyclic shift register with only one set bit may cyclically generate an enable signal for each comparator. The comparators may be used to match the current time of each simulated mRNA molecule by halting the fastest mRNA when the time difference exceeds a parametric threshold.

In other words, instead of halting the entire system to approximate causality, we can halt only consecutive pairs. In this way, only one comparator may be active at each time. That results at most in only one halted mRNA molecule at each time. The threshold input to the comparator can also determine the dT inaccuracy that we can tolerate. In practice, this solution is less accurate than the mRNA synchronization solution. In the final iterative model presented in the article, we used the mRNA synchronization method, and the performance speedup was quite sufficient for our needs. Implementing the match-in-pairs method should be considered if we can tolerate a less accurate model that runs faster.

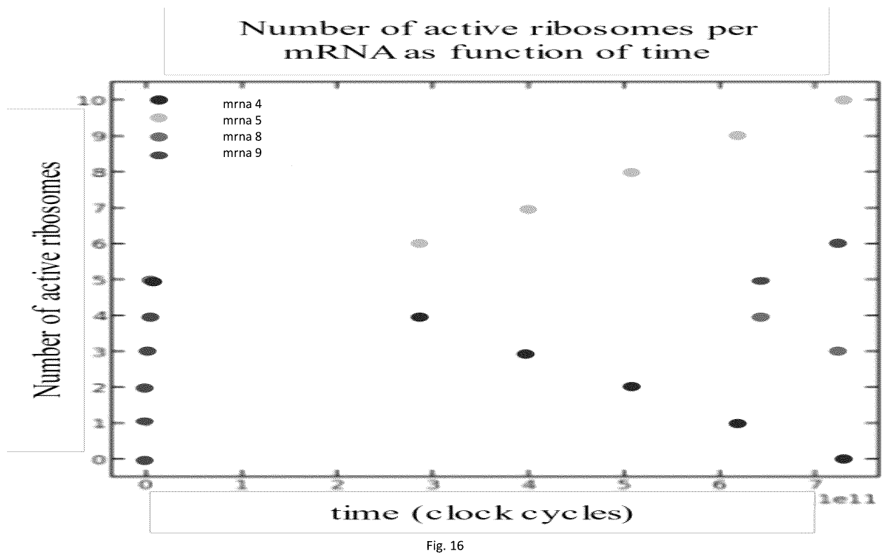

Reference is now made to FIG. 16, which is a graph showing the number of active ribosomes on each mRNA molecule as function of time (in nano seconds) in the initial model for selected mRNA molecules. As shown in FIG. 16, there is a bias of the ribosome allocation probability towards consecutive mRNAs.

The concatenation of the mRNA ribosomes' FIFOs causes a bias in the free ribosome allocation probability. The first mRNA molecules will always receive a ribosome molecule at the beginning of the simulation. Moreover, when a ribosome is released, the next mRNA molecule will have priority in receiving it. In a real cell, the allocation of free ribosomes happens randomly with uniform probability. The assumption was that in the steady state, the variance in the mRNA lengths will cause enough randomness so that the free ribosomes will be distributed uniformly among the mRNA molecules and this bias will not be noticeable. In fact, by analyzing the simulation results, the bias of the ribosomes allocation is not neglectable and shown in FIG. 16.

INITIAL DESIGN—CONCLUSIONS. For conclusion, here is the list of the insights that we got from the initial design analysis:

(1) In case we have a memory bottleneck, we should consider storing the codon delays in two separate concatenated memories instead of a single large one. That was useful in the iterative model and as shown in the main text, the codon's data is stored in two concatenated memories. In the parallel model, the bottleneck was logic utilization and not memory utilization. Therefore, in the parallel we kept the single large memory for the codon data for the state machine simplicity.

(2) Working with absolute indexes for the ribosomes leads to a large memory consumption. The ID of the ribosomes is not important for the sake of mRNA translation modelling. Therefore, in the iterative model, we kept the ribosomes' state inside a FIFO and the only thing that we kept track of was the number of active ribosomes (and their order—which is enforced by using a FIFO).

(3) When having autonomous separate mRNA molecules, it is important to make sure that the local time of each mRNA molecule is synchronized with all other mRNAs. In the parallel model it is given by having the system run simultaneously in parallel and not iteratively. In the iterative model, as shown in the article, we synchronized all mRNA state machines by adding a “hold” signal that releases only if all mRNAs are done with the current iteration over their ribosomes.

(4) The global arbiter architecture should be embraced to avoid bias in the ribosomes' distribution among mRNA molecules.

The general idea is to avoid iterating over each active ribosome of a single mRNA molecule. Instead, we shall try having the ribosomes act as independent hardware entities. As suggested above, the mRNAs are connected to a global arbiter. The arbiter receives the request & release signals of the mRNAs and generates grants the ribosomes. The arbiter in the new design contains the global counter of free ribosomes.

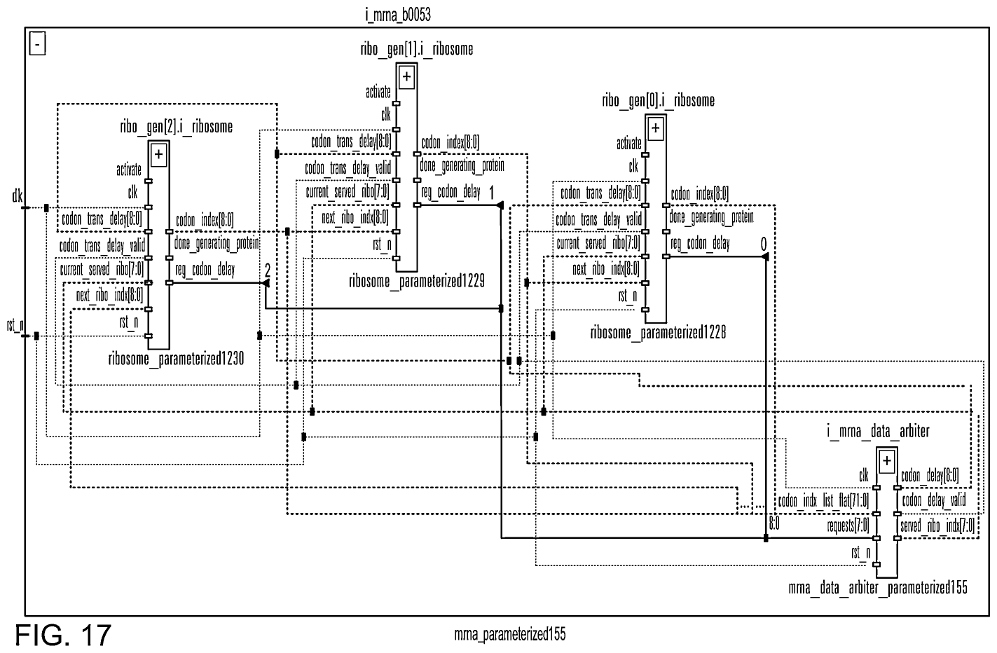

Reference is now made to FIG. 17, which is a block diagram depicting connections between resource modules (here three depicted hardware ribosomes) 210 and a global data arbiter 30, according to some embodiments of the invention.

As elaborated herein (e.g., in relation to FIG. 5), and shown in FIG. 17, each mRNA molecule 20, may include a concatenated structure 240 of hardware implemented resource modules (e.g., ribosomes) 210.

Each ribosome 210 has its own state machine 210SM. Each ribosome 20 may be connected to the index of its subsequent ribosome in concatenated structure 240 (to make sure it can skip to the next codon when done counting). The last ribosome 210 in the hardware structure is connected to the index of the first in a cyclic manner.

One can think of the concatenated ribosomes 210 as a hardware ribosome FIFO. First, all the ribosomes start inactive and when the global arbiter 30 assigns a new ribosome 210, the mRNA 20 logic is responsible for activating a ribosome 210 in the hardware. The mRNA logic 20 is responsible for keeping the following values:
(1) Read pointer—a pointer to the first current active ribosome;
(2) Write pointer—a pointer to the first inactive ribosome—when a new ribosome is granted by the global arbiter, the one pointed by the write pointer is activated;
(3) First pointer—a pointer for the first active ribosome from the 5′ end of the mRNA molecule—that is kept for the mRNA logic to determine if a new ribosome request should be issued; and
(4) Active ribosomes counter—to make sure that the hardware ribosomes FIFO does not saturate.

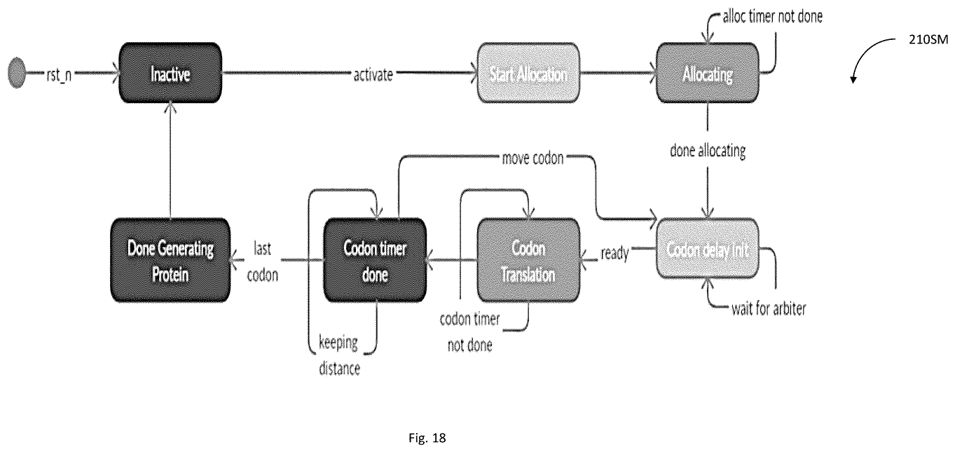

Reference is now made to FIG. 18, which is a block diagram depicting an example of a state machine 210SM of a resource (e.g., ribosome) module 210, according to some embodiments of the invention. As shown in FIG. 18 state machine 210SM may begin in an inactive or idle state, and then continue to the allocation states when it receives the activate signal from the mRNA module. Next, after the allocation timer is done (done_allocating), the ribosome state machine 210SM may continue with translating the codons. It is done by first retrieving the codons' delay from the data arbiter (codon_delay_init) and then decrementing the translation timer. When the translation timer reaches 0, the ribosome state machine 210SM may until the concatenated ribosome is far enough (keeping_distance). When the index of the current codon is equal to the simulated mRNAs length (e.g., number of simulated codons), the simulated ribosome's translation task may be done (e.g., a simulated protein may be generated.

As elaborated herein, system 100 may be configured such that all resource (ribosome) modules 210 may operate freely, and maintain the minimal distance from each other. To get the wait time for a given simulated codon index, we could have kept a local BRAM memory for each ribosome in hardware. This may not be efficient in respect to hardware utilization, since the resource (ribosome) modules 210 may spend most of the time counting. The event of skipping to the next codon occurs far less than timer advancement. Therefore, we shall have the mRNA codon delay table in a common place accessible for all hardware ribosomes. This memory should be managed by an arbiter. We implemented it by a simple round-robin arbiter. The arbiter iterates over all ribosomes' indexes (including the inactive ones) and outputs the delay value. The ribosomes contain a comparator that constantly examines if their index is the one being served (Instead of having the arbiter signaling each endpoint—that would have cost more hardware).

The parallel design was implemented using the Zynq Ultrascale+ chip. To fit in as many mRNAs and ribosomes as possible, we had to make some adjustments. First, notice that the true bottleneck of the entire design is the size of a single hardware ribosome. The internal state of the ribosome is not expected to be different than in size from the iterative design. As before, we may keep for each resource module (e.g., ribosome):(1) The index of the current codon; (2) The remaining allocation time; (3) The remaining translation time; and (4) The resource module's state. This state may be for example one of an allocating state (e.g., when the ribosome 210 is attaching to mRNA 20), a counting state (e.g., when the ribosome 210 is translating a codon, an advancing state (e.g., when the ribosome 210 is advancing from one codon to another, and a done state (e.g., when the ribosome 210 is done translating, and is being deallocated or dissociated from mRNA module 20).

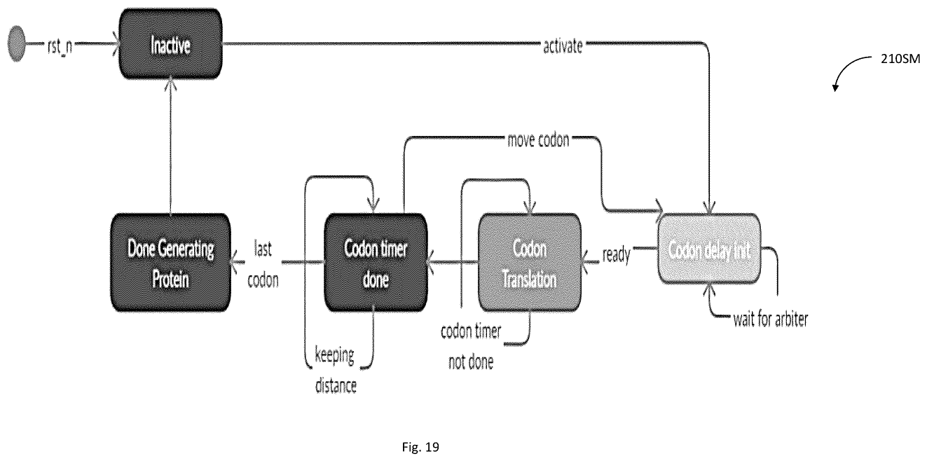

Reference is also made to FIG. 19, which is a block diagram depicting an example of a state machine 210SM of a resource (e.g., ribosome) module, according to some embodiments of the invention. As shown in FIG. 19, state machine 210SM may be equivalent to that depicted in FIG. 18, e.g., having the same states and signals, except the allocation states that may be removed. That is done by placing the allocation logic as part of the client (mRNA) module 20 to save resources as elaborated herein.

The difference in the parallel design is that each ribosome 210 is implemented in hardware and may run autonomously. That means that we also may keep that state machine logic 210SM & adders for each ribosome. Also, each mRNA molecule 20A (as before) should keep a buffer of hardware ribosomes 210. Theoretically, the buffer size for a mRNA molecule of size M and ribosome size D (minimal distance) is M/D. Although that is the theoretical bound, the practical value of maximal ribosomes active on a single mRNA depends on the global availability of ribosomes in the cell.

Let us denote the global number of ribosomes as R and the number of mRNAs in the cell as N. Then, if the ribosomes' allocation happens uniformly, it is not probable that a single mRNA molecule will have substantially more than R/N active ribosomes (In the “methods” section we examined the dependency on the length of the mRNA molecule with respect to others in the simulation).

So, the size of the hardware ribosomes FIFO for each mRNA molecule was first chosen to be min (M/D, 2R/N).

Moreover, notice that at a given time, only one ribosome can be in the allocation state. That means that if the allocation state machine logic & allocation timer are substantial, we can consider extracting the allocation from the internal ribosome state machine to the mRNA module level. The resulting state machine of the ribosome will then be as depicted in FIG. 19.

As explained, the Zynq core 110 may communicate with FPGA PL 120 via the AXI interface 130. This interface may reveal a set of configuration registers that are used for activating the model and reading back the results. We created a compact list of registers for the configuration. An example of implementation of these interface registers is as follows. The base address of our module in the ARM 110 memory space is 0xA000000

| Register name | Register Address | Direction |
|---|---|---|
| CTRL_REG | 0xA0000000 | Write |

0: model_rst_n - reset the iterative model.
1: model_config_rst_n - reset the model configuration.
2: model_config_enable - enable the model configuration.
3: stop_time_config_enable - enable the stopping time configuration.
4: clear_interrupt - when written 1, the iterative model clears the interrupt.

| | | |
|---|---|---|
| MRNA_CONF_ADDR | 0xA0000004 | Write |

[31:16] - mrna_index - the index of the addressed mRNA for codon
configuration
[15:0] - codon_index - the index of the configured codon in the pointed
mRNA

| | | |
|---|---|---|
| MRNA_CONF_DATA | 0xA0000008 | Write |

[5:0] - new codon code to be written. Notice: this value may only written if the
model_config_enable is on.

| | | |
|---|---|---|
| MODEL_STOP_TIME | 0xA000000C | Write |

[31:0] - the stop time in milliseconds - when the iterative model finish modelling the specified
number of milliseconds, the model_done interrupt goes high.

| | | |
|---|---|---|
| PROT_MRNA_ADDR | 0XA0000010 | Write |

[31:0] - the index of the mRNA from which we wish to read the protein counter. Notice that in
practice, only the last 10 bits are used because we currently support 1024 mRNAs.

| | | |
|---|---|---|
| MODEL_REAL_TIME | 0XA0000014 | Read |

[31:0] - the current model time in milliseconds.

| | | |
|---|---|---|
| PROT_CNTR | 0XA0000018 | Read |

[31:0] Contains the protein counter of the mRNA pointed by PROT_MRNA_ADDR.

| | | |
|---|---|---|
| SEL_MRNA | 0XA000001C | Read |

[31:0] Connected to the internal pipelined mux of the protein readback. Therefore, it is
important to compare this value to PROT_MRNA_ADDR before reading the PROT_CNTR register.

| | | |
|---|---|---|
| STATUS_REG | 0XA0000020 | Read |

0: model_done - that bit has the same value as the interrupt signal model_done

| | | |
|---|---|---|
| CURR_FREE_RIBOS | 0XA0000024 | Read |

[31:0] - The number of currently free ribosomes in the cell.

| | | |
|---|---|---|
| MAX_CELL_RIBOS | 0XA0000028 | Write |

[31:0] - assign the total number of ribosomes in the cell at the beginning of the run.

To facilitate the access to the above low-level interface of the configuration registers, we developed a dedicated Python package for operating the FPGA PL 120. Inside this package, the PL_CONTROL class is defined. The constructor of this class only receives the BAR (base address) of the iterative model wrapper in the ARM address space. Then, the class uses the MMIO class from the PYNQ package to access the above list of objects. When instantiating the class, the created object exports the following methods:
assert_model_reset( )—resets the iterative model.
deassert_model_reset( )—de-asserts the model reset and by that, letting it run until the configured stopping time
assert/deassert_config_reset( )—asserts/de-asserts the configuration reset to allow the model configuration before running it
config_model_time (stopping time)—This function configures the stopping time of the simulation (receives the required value as an argument)
set_number_of_ribosomes (num_ribosomes)—configures the number of ribosomes in the cell to the received value
config_mrna_codon (mrna_indx, codon_indx, codon_code)—this function configures the codon_indx codon in the mrna_indx mRNA with codon_code value
read_protein_counter (mrna_indx)—this function returns the current generated proteins counter of the required mRNA index
read_num_free_ribos( )—this function returns the current number of free ribosomes in the model.

print_hardware_status( )—prints basic information regarding the iterative model status: the model_done signal status, the current model time, the current number of free ribosomes and the number of generated proteins for each mRNA up to this point. The following is an exemplary list of parameters for the chip module.

| Parameter | Description | Nominal values |
|---|---|---|
| P_NUM_MRNAS | This parameter dictates the amount | May always be a power of 2. Also, |
| | of mRNA molecules instantiated | currently, the uniform arbiter |
| | in the top module. | supports up to 1024 endpoints, so |
| | | this value may not exceed 1024 |
| | | without updating the uniform |
| | | arbiter. |
| P_GLOBAL_TIME_{—} | The bit width of the local timer of | Up to 32 bits (as that is the width of |
| WIDTH | each mRNA molecule. | the AXI registers). |
| P_FREE_RIBOS_{—} | The width of the ribosomes | Also should be up to 32 bits as that |
| COUNTER_WIDTH | counter. | is the width of the AXI registers. |
| P_GENERATED_PROTS_{—} | The width of the local proteins | For the POC, 10 bits suffices. This |
| COUNTER_WIDTH | counter for each mRNA molecule. | value should also be kept bellow 32. |

Next, here is the list of the interface signals of the module:

| Signal name | Direction | Type | Description |
|---|---|---|---|
| clk | Input | Clock | That is the clock signal for the model. |
| | | | Currently, the maximal frequency allowed |
| | | | is up to 200 MHz. |
| rst_n | Input | Reset | That is the reset signal for the model. |
| conf_clk | Input | Clock | That is the configuration clock - used to |
| | | | configure the mRNA memories, the |
| | | | stopping time and also the number of |
| | | | ribosomes in the cell. It is also used to |
| | | | sample busses that are later copied to the |
| | | | AXI registers. |
| config_rst_n | Input | Reset | Configuration reset signal. Resets the |
| | | | configuration registers and logic (like the |
| | | | stopping time register). |
| memory_config_{—} | Input | Enable | When 1, the module configures the mRNA |
| enable | | | codon's list with the supplied values. |
| config_mrna_indx | Input | Bus | That is the address of the mRNA |
| | | | molecules that is about to be configured |
| | | | (its codons' ROM is about to be updated). |
| config_codon_indx | Input | Bus | The index of the codon in the pointed |
| | | | mRNA (via config_mrna_indx) that is |
| | | | going to updated in the codon's ROM. |
| config_codon_data | Input | Bus | The new value of the configured codon. |
| stop_time_config_{—} | Input | Enable | When 1, this signal enables the update the |
| enable | | | model stopping time. |
| stop_time | Input | Bus | The required value of the model stopping |
| | | | time. |
| num_ribosomes | Output | Bus | The assigned number of ribosomes for the |
| | | | current model run. |
| real_time_ms | Output | Bus | The current model time in milliseconds (in |
| | | | real cell time). |
| porteins_counters_{—} | Output | Bus | This is a flatten bus which contains the |
| flat | | | concatenated protein counters of all |
| | | | mRNA molecules. That is used by the |
| | | | wide multiplexer in the AXI wrapper |
| | | | (described further herein). |
| model_done | Output | Interrupt | This is the output interrupt that the model |
| | | | raises when reaching the configured |
| | | | stopping time. |
| free_ribosomes_{—} | Output | Bus | The current value of free ribosomes in the |
| counter | | | cell. |

Also, the module contains the or_release_ribo_list and or_release_ribo_from_mrna_list for debug purposes. Moreover, apart from containing the instantiations of the mRNA molecules and global ribosome, this module is also responsible for sampling the stopping time and generating the mrna_wr_en signals for all mRNAs. This signal is calculated in the following generate block example:

genvar g_indx; generate
for (g_indx=0; g_indx < P_NUM_MRNAS; g_indx=g_indx+1)
assign mrna_conf_wr_en_list[g_indx] = memory_config_enable
& (g_indx == config_mrna_indx);
endgenerate

Furthermore, the current time of the model is calculated in all mRNA molecules simultaneously. For the sake of generating the model_done interrupt signal, the top module uses the current time port of the first mRNA (arbitrarily). The synthesis process recognizes that the other counters are not in use and deletes their instantiation to reduce the utilization. That is done for the sake of simplicity in the routing and instantiation of the mRNA molecules. Also, the top module also contains the generation of the hold signal which is shared between all mRNAs to keep the state machine synchronization. This signal is only used in the iterative model since the parallel model is synchronized in its nature. At the beginning, the hold generation was simply done by the negation of the bitwise and of the ready_for_next_iteration signals from all mRNAs as follows:

wire [P_NUM_MRNAS − 1 : 0] done_iter_list;
wire hold;
assign hold = !(&done_iter_list);

Later, to ease the router's task to meet the timing constraints, this wide and gate was replaced by a pipelined version of it for the case of 1024 mRNAs, as in the following example:

wire [1023 : 0] done_iter_list;
reg [1:0] and_done_iter_list; wire hold;
always @(posedge clk) begin
and_done_iter_list[0] <= &(done_iter_list[511:0]); and_done_iter_list[1] <=
&(done_iter_list[1023:512]);
end
assign hold = !(&and_done_iter_list);

Additionally, before the instantiation of the global arbiter, the module may contain sample logic for sampling the free ribosomes counter at the exact moment in which the model_done signal goes high.

ROUND-ROBIN GLOBAL ARBITER. The round-robin arbiter is a version of the global arbiter. Apart from having the P_NUM_MRNAS parameter as before, it may also have the following parameters:

| Parameter | Description | Nominal values |
|---|---|---|
| P_NUM_RIBOS | The number of ribosomes in the | This module may not be used in the |
| | cell. This value initializes the | final POC of the system, so the value |
| | internal free ribosomes' pool | is hard coded by this parameter. |
| | counter. | Having this value configurable as in |
| | | the uniform arbiter is easy and can be |
| | | done in the same way. |
| P_MAX_FREE_RIBOS | Contains the maximal amount of | The main text includes an explanation |
| | release events that can occur | on how this value was calculated. |
| | between consecutive visits of | |
| | the arbiter in the same mRNA | |
| | molecule. | |

The interface of the module consists of the clk and rst_n signals as before and the buses mrna_request, mrns_release and mrna grant which are all of width P_NUM_MRNAS and contain the request and release signals of all mRNAs and the output grant signal for all mRNAs. Notice that later, this interface was changed for the uniform arbiter to avoid the demultiplexer for the mrna_grant bus. The module contains a simple counter of the free ribosomes in the cell. The counter is initialized via the P_NUM_RIBOS parameter, and is decreased upon granting a ribosome. This value is increased when the arbiter lands on an mRNA molecule that has released ribosomes from the last time the arbiter visited that mRNA. That means that the global arbiter module should keep a local counter, for each mRNA molecule, that counts the number of ribosomal release events until the next time it visits the same mRNA. The width of that counter is the P_MAX_FREED_RIBOS parameter (its calculation is explained in the main text). This may be done by the following hardware description code block example:

genvar g_indx; generate
for (g_indx=0; g_indx < P_NUM_MRNAS; g_indx=g_indx+1) begin always
@(posedge clk) begin
if (~rst_n)
freed_counter[g_indx] <= {P_MAX_FREED_RIBOS{1′b0}}; else
freed_counter[g_indx] <= (current_mrna == g_indx)
? {P_MAX_FREED_RIBOS{1′b0}}
: freed_counter[g_indx] +
mrna_release[g_indx];
end
end endgenerate

In this code, the freed_counter of each mRNA molecule is cleared whenever the arbiter is currently visiting this mRNA. Then, the new value of the free ribosome counter may be calculated as in the following example:

always @(posedge clk) begin if (~rst_n)
free_ribos_counter <= P_NUM_RIBOS; else
free_ribos_counter <= free_ribos_counter
+ freed_counter[current_mrna]
− (mrna_request[current_mrna] & available_ribos)
+ mrna_release[current_mrna];
end

In this block, the new value of the free_ribos_counter is calculated by adding the current value of the local release counter of each mRNA to the current value, decreasing the counter by 1 if the current mRNA requests ribosomes (and there are ribosomes available) and increased by one if it happens to be a clock cycle in which a ribosome is released from the current mRNA.

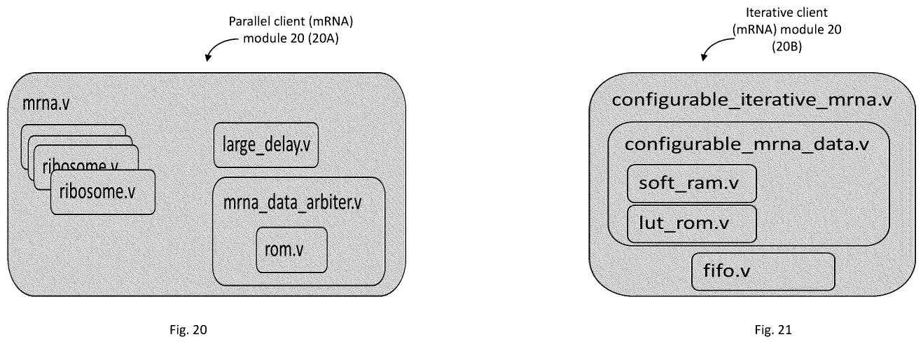

Reference is now made to FIG. 20, which is a block diagram depicting HDL modules' hierarchy of the parallel client (mRNA) module 20A, according to some embodiments of the invention.

As elaborated herein, parallel client (mRNA) module 20A this module consists of a concatenated structure of hardware ribosomes (ribosome.v). This module also contains the codon's data inside a ROM (rom.v) that is controlled via an arbiter that servers all ribosomes (mrna_data_arbiter.v). Let us begin by first explaining the structure of each module and then we proceed with the high-level module interface and parameters.

Hardware ribosome module. The hardware ribosome 210 essentially consists of the state machine 210SM module. This module receives the following parameters:

| Parameter | Description | Nominal values |
|---|---|---|
| P_MRNA_LENGTH | The length of the mRNA molecule (in | Positive integer. |
| | codons). | |
| P_CODON_INDX_WIDTH | The width of the index to the codons in | ┌log(P_MRNA_LENGTH)┐ |
| | the specific mRNA. This value depends | |
| | on the length of the mRNA and therefore | |
| | parametric. | |
| P_CODON_DELAY_WIDTH | The width of the translation delay timer | This value may be |
| | of the codons. | calculated according to |
| | | the maximal delay of the |
| | | slowest codon. |
| P_RIBO_MIN_DISTANCE | The minimal distance (in codons) that | Positive integer. |
| | should be kept between two consecutive | |
| | ribosomes). | |
| P_RIBO_ALLOC_DELAY | The allocation time for the specific | Positive integer. |
| | mRNA. As shown in the main text, this | |
| | value may be used when the allocation | |
| | delay is part of the ribosomes' state | |
| | machine. | |
| P_RIBO_INDX | The index of the current ribosome in the | From 0 to the number of |
| | hardware ribosomal chain. This value is | ribosomes in the local |
| | used to check if the current served | mRNA (excluding). |
| | ribosome by the data arbiter is the local | |
| | ribosome. | |
| P_MAX_ACTIVE_RIBOS | Number of hardware ribosomes in the | Positive integer. |
| | current mRNA. | |

Those parameters are automatically assigned to the ribosome module 210 via a large ‘generate’ block inside the mrna.v module. That basically means that those parameters are either propagated from the mrna.v parameters or are generated using the generate variable. As mentioned in the top module section, the parameters for mRNA modules 20 are generated via the python script in accordance with the various parameters of each mRNA in E. coli. Next, here are the non-trivial ports for the ribosome module:

| Signal name | Direction | Type | Description |
|---|---|---|---|
| activate | Input | Control | This signal is generated by the client (mRNA) |
| | | | module's 20 state machine 20SM. When the |
| | | | mRNA receives a new ribosome 210 from the |
| | | | global arbiter 30, the mRNA state machine 20SM |
| | | | may activate the next IDLE hardware |
| | | | Ribosome 210. This signal enables the ribosome's |
| | | | state machine 210SM. |
| next_ribo_indx | Input | Bus | This is the connection to the codon index of the |
| | | | consecutive ribosome. This signal may be used to |
| | | | keep the minimal distance between the ribosomes. |
| codon_indx | Output | Bus | The index of the current codon. This bus may be |
| | | | connected to the data arbiter of the mRNA used to |
| | | | request the translation delay of the current codon |
| | | | from ROM 230. |
| | | | This bus may also be connected to the next |
| | | | hardware ribosome's next_ribo_indx bus. |
| req_codon_delay | Output | Control | When 1, the ribosome waits for the arbiter to send |
| | | | the delay of the current codon from the ROM |
| | | | 230. |
| codon_trans_delay | Input | Bus | The value of the translation delay from the ROM |
| | | | 230 arbiter (e.g., round-robin arbiter 220). |
| current_serverd_ribo | Input | Bus | The index of the current served ribosome by the |
| | | | client's (mRNA) 20 ROM arbiter (e.g., round- |
| | | | robin arbiter 220). |
| codon_trans_delay_valid | Input | Control | This is the valid signal for the |
| | | | current_served_ribo |
| | | | and codon_trans_delay busses. |
| done_generating_{—} | Output | Control | When 1, the simulated ribosome finished |
| protein | | | generating the simulated protein of the current |
| | | | simulated mRNA strand. After raising this |
| | | | signal, the resource (ribosome) state machine |
| | | | 210SM goes to IDLE state until re- activated. |

Apart from the state machine 210SM, it is important to understand the following aspects of resource (ribosome) module 210. For example, the done_generating_protein signal can be 1 only for one single hardware ribosome (for each mRNA). That is because the hardware ribosomes 210 are concatenated, and it is not possible for a ribosome 210 to generate a protein if it is not the current first active ribosome. Also, the next_ribo_indx may be wired to the concatenated ribosome 210. This signal is important for keeping the required distance from the consecutive ribosome 210. This bus is used to generate the clear_to_move signal that signals the ribosome's state machine that the ribosome can advance to the next codon. This signal may be generated as elaborated in the hardware description code below:

wire clear_to_move;
assign clear_to_move = (next_ribo_indx <= codon_index) ? 1′b1
: (next_ribo_indx − codon_index > P_RIBO_MIN_DISTANCE);

In this code, system 100 may check whether the current resource (ribosome) module 210 represents the first simulated ribosome on the simulated mRNA strand. The check is done by comparing the current index to the next ribosome index. If so, then the clear_to_move signal may be activated. Otherwise, resource (ribosome) module 210 can advance the current codon index only if the distance between this ribosome to the next is bigger than P_RIBO_MIN_DISTANCE parameter (see the parameters table).

Also, to keep the ribosome 210 as minimal as possible, it is important to make sure that the coding of the states in the state machine allows compact usage of the LUTs. For example, let us view the following code in the ribosome module:

always @(posedge clk) begin if (~rst_n)
codon_delay_timer <= {P_CODON_DELAY_WIDTH{1′b0}}; else
codon_delay_timer <= (current_state == S_CODON_TIMER_INIT)
? codon_trans_delay : codon_delay_timer − 1′b1;
end

Here we can see that the delay timer of the current codon is handled. We can see that if the ribosome's state machine 210SM is in the initialization state, the timer is initialized to the value that eventually comes from the ROM's arbiter. Otherwise, the timer decrements by 1. The comparison of the state register current_state to the required state (S_CODON_TIME_INIT) might consume more resources if the states are not coded properly. In this case for instance, if we use one-hot coding for the states, the comparison current_state==S_CODON_TIMER_INIT will be equivalent to just a single bit in the state register.

Large delay module. This module is used for delaying the done_generating_protein signal for modelling the diffusion property of the ribosomes. The diffusion property basically means that it takes time for a ribosome to be available again by the cell after it is released from the mRNA molecule. The diffusion delay in clock cycles is chosen to be the value that causes some percentage of ribosomes to be in diffusion state in steady state. For E. coli, nominally there are 30% of the ribosomes in diffusion in the steady state. Therefore, the delay value is calculated as follows:

$$diffusion delay = \frac{{\sum }_{i = 0}^{M - 1} ( {A}_{i} + {\sum }_{j = 0}^{{L}_{i} - 1} {c}_{j}^{i} )}{M} ( \frac{D}{1 - D} )$$

M is the number of mRNA molecules, Aᵢ is the allocation delay of the i-th mRNA, Lᵢ is the length of the i-th mRNA, cⁱ is the translation delay of the j-th codon of the i-th mRNA and D is the diffusion factor (0.3 for 30%). This formula basically calculates the average time it takes for an mRNA molecule to be translated (ignoring the traffic jams) and uses it to calculate the time a ribosome should wait to receive the required diffusion percentage. This formula is an approximation (because it ignores the ribosomes' traffic jams) but it yields the required results in practice.

Using this formula, in E. coli, we get that the diffusion time is between 20,000 milliseconds and 40,000 milliseconds. This value is quite large. The most straight forward implementation of a delaying module is a simple shift register. In this case, a shift register may be of length of 30,000 registers on average. This shift register needs to be duplicated for the number of mRNA molecules in the design. So, for 1024 mRNAs, if we use a simple shift register, it will consume 30,000*1024=30.72 million registers. In an exemplary FPGA chip, we only have 460,800 flip-flops. Since we cannot afford a simple shift register, we then considered using the SLICEM slices in the Zynq FPGA. Those specific slices allow using the LUT's internal memory as a shift register.

In our FPGA, we have 6 input LUTs. Therefore, in each LUT we can store up to 26=64 bits of our shift register. In ZCU104 we have 101,760 LUTs that can be used as shift registers. Therefore, we can store up to 101,760*64=6,512,640 bits of shift registers. Therefore, also when using the SLICEM LUTs, we do not have enough resources for implementing the diffusion delay as shift-registers. Therefore, we developed the large_delay.v module that utilizes the specific features of delaying the release signal of a ribosome from an mRNA molecule. The module has two parameters:
P_NUM_FAST_CLK_CYCLES—this parameter is the value of the required diffusion delay in clock cycles. As mentioned, for E. Coli it should be around 30,000.
P_NUM_CLK_CYCLES_PER_ITER—this parameter is used to define an internal counter that generates a slow enable signal.

Then, the module delays the fast input signal by approximately P_NUM_FAST_CLK_CYCLES. It is done by an internal shift register of size P_NUM_FAST_CLK_CYCLES/P_NUM_CLK_CYCLES_PER_ITER that advances each P_NUM_CLK_CYCLES_PER_ITER. There are certain points that should be mentioned here. The first, is that the value of the parameters may be chosen as such that no release event of a ribosome can occur in the P_NUM_CLK_CYCLES_PER_ITER consecutive clock cycles after the previous ribosome release. That utilizes the fact that ribosomes are released at a rate that is bounded by the time it take to translate the last 9 codons (the minimal distance in E. coli).

The second point is that the fast_signal input to the module (the release signal) is asynchronous to the local iteration counter and is kept until the next iteration begins. That causes a slight variation in the diffusion delay (typically around half P_NUM_CLK_CYCLES_PER_ITER).

As mentioned in the main text, instead of copying the codons' delay for each ribosome, a single ROM may be kept and arbitered using a simple round robin arbiter that serves all hardware ribosomes. That is implemented in the mrna_data_arbiter module. This module may contain the actual data of the mRNA's codons. As shown in the previous sections, we found that it is more memory efficient to keep the codon's data in two concatenated memories instead of a large, big one. For the parallel model, the memory was not a bottleneck for the utilization, so we kept a single large table from the codon index to the codon delay. The table is stored in a ROM memory (in rom.v) module. The ROM is initialized using a “.mem” file that is generated by the Python script that also instantiate the mRNA modules as the path to that configuration file is a parameter of the mRNA module and eventually propagates to the ROM module. Inside the ROM module the following code is stored with directives to the synthesis tool, as in the example below:

(* rom_style = “block” *) reg [DATA_WIDTH−1:0] mem [0:MEM_SIZE];
initial begin
$readmemb(INIT_FILE, mem, 0, ACTUAL_MEM_SIZE−1);
end

In this hardware description code, the rom_style directive that direct the synthesis tool to implement the ROM as a BRAM is first seen. That is important because in the parallel case the memory is not the bottleneck. If we emit this directive, the synthesis might implement the ROM as distributed RAM and that will cost LUTs which are the bottleneck of the parallel design. Also, in this code we can see the $readmemb directive that receives the INIT_FILE path (the .mem file). This directive directs the synthesis to initialize the BRAM in hardware with the contents of the given INIT_FILE. The format of the file is simply a list of binary encoded values of the bits inside the ROM. If one wished to save more disk files for the storage of those memory files, it is possible to use the $readmemh directive that receives files with data that is encoded in hexadecimal.

PARALLEL MRNA MODULE—PARAMETERS AND INTERFACE. As mentioned, the mRNA modules are parametric and the python script that instantiate them in the top module is responsible for setting the parameters. The parameters: P_CODON_INDX_WIDTH, P_CODON_DELAY_WIDTH, P_RIBO_MIN_DISTANCE, P_MAX_ACTIVE_RIBOS, P_RIBO_ALLOC_DELAY P_MRNA_LENGTH are the same as presented in the ribosome module. The parameters P_DEFUSION_TIME and P_DEFUSION_ITERATION_TIME are the parameters for the large_delay module presented earlier. P_MRNA_INIT_FILE is the path of the “.mem” file used for initializing the internal ROM. The interface of the module is very simple and is the same as shown in the high-level block diagram in the main text. Apart from the trivial signals (clocks and reset), the interface consists of the following:

| Signal name | Direction | Type | Description |
|---|---|---|---|
| req_ribo | Output | Control | When 1, the mRNA is free to receive a new |
| | | | ribosome from the pool. |
| release_ribo_to_pool | Output | Control | When 1, the mRNA releases the ribosome to the |
| | | | global pool after diffusion. |
| release_ribo_from_mrna | Output | Control | When 1, a new protein is generated but the |
| | | | ribosome is first entering the diffusion state. |
| | | | This signal is delayed via the large_delay |
| | | | module to produce the |
| | | | release_ribo_to_pool output. |
| ribo_granted | Input | Control | When 1, the global arbiter grants a new |
| | | | ribosome to the mRNA molecule. |

Notice that the ribo_granted signal is replaced with the mRNA index, that is currently served, in the final POC with the iterative mRNA. This simple adjustment can be applied here if necessary.

Reference is now made to FIG. 21, which is a block diagram depicting HDL modules' hierarchy of the configurable iterative client (mRNA) module 20B, according to some embodiments of the invention.

As elaborated herein, iterative client (mRNA) module 20B may include a FIFO that is responsible for keeping the state of the ribosomes. The configurable_mrna_data module contains the codons' data and as the name suggests, this module also support reconfiguration of the codons' table. This module also contains two state machines as shown in the main text.

The iterative mRNA data module may be responsible for keeping and reconfiguring the mRNA's codon data. In the parallel model, the codons' data may be stored inside the data arbiter module. Here, there is only one consumer for the codons' data—the mRNA state machine. Therefore, the arbiter is not required here. As opposed to the parallel model, in which the bottleneck was the logic utilization, the bottleneck in the iterative model is the memory utilization. Therefore, we may apply the two-table method that was described herein. The mapping between the codons' code to the codon data is stored in lut_rom.v. This is the exact same module as the rom.v module, that was part of the parallel models' data arbiter, apart from a small change:

(*rom_style=“distributed”*)reg[DATA_WIDTH-1:0]mem[0:MEM_SIZE];

The change is in the synthesis directive—instead of using the “block” directive, we use the “distributed”. That directs the synthesis to favor the implementation of that memory using LUTs instead of BRAMs. As shown earlier, the synthesis indeed implements this memory using 16 LUTs as desired. Also, notice that the initialization of this memory may be generated. As before, the $readmemb directive is used with the codons' delay values.

Next, the mapping between the codon index to the codons' code is stored in the soft_ram.v module. This module is like the ROM module. Its interface also includes the write logic required for updating the values. Also, the memory register declaration is as follows:

reg[DATA_WIDTH-1:0]mem[0:MEM_SIZE];

Here we see that no specific directive was given to the synthesis. We found that this degree of freedom makes a difference and allows fitting more mRNAs in the design as the synthesis can implement small mRNAs using distributed memory and large mRNAs using BRAMs. Also, this memory begins with an empty cell (initialized to 0). That is done to have the state machine treat the allocation delay as all other codons. The reason why we did not simply put the allocation delay as the first value in that memory is because the allocation delay is typically large and therefore that memory would have been unnecessarily wide.

Parameters of iterative client (mRNA) module 20B include P_CODON_INDX_WIDTH, P_CODON_DELAY_WIDTH, P_RIBO_MIN_DISTANCE, P_MAX_ACTIVE_RIBOS, P_RIBO_ALLOC_DELAY, P_MRNA_LENGTH, P_DEFUSION_TIME and P_DEFUSION_ITERATION_TIME, which are the same as elaborated above. Additional iterative client (mRNA) module 20B parameters include:

| Parameter | Description | Nominal values |
|---|---|---|
| P_MRNA_INDX | The index of the current mRNA | Up to the number of mRNA |
| | module. This may be used when the | molecules in the design. |
| | arbiter outputs the index of the mRNA | |
| | that is currently served. | |
| P_MRNA_INDX_WIDTH | The width in bits of the mRNA index | ┌log(NUM MRNAS)┐ |
| | param. | |
| P_CODON_WIDTH | The width of the codons' code. | 6 bits. |
| P_DELAY_WIDTH | The maximal width of the | Positive integer. |
| | ribosomes' timer that is used for the | |
| | allocation delay and for the codons' | |
| | delay. | |
| P_ALLOC_DELAY_{—} | The width of the allocation delay. | Positive integer. |
| WIDTH | | |
| P_RIBO_FIFO_ADDR_{—} | The address width of the FIFO used to | Ceil value of the log of the |
| WIDTH | store the ribosomes' state. | maximal amount of |
| | | simultaneously active |
| | | ribosomes on the |
| | | current mRNA. |
| P_LOCAL_TIME_{—} | The width of the local time counter. | Positive integer (up to 32 |
| WIDTH | | bits to be read by the |
| | | AXI registers). |
| P_CODONS_MEM_FILE | Path to the .mem file used to initialize | File path. |
| | the lut_rom module with | |
| | the codons delay values. | |
| P_CODONS_DELAY_{—} | Path to the .mem file used to | File path. |
| FILE | initialize the soft_rom module with | |
| | the specific mRNA's codons' list. | |
| P_GENERATED_PROTS_{—} | The width of the local protein counter. | Up to 32 bits (eventually |
| CNT_WIDTH | | read by the AXI |
| | | registers). |

Next, the interface of iterative client (mRNA) module 20B may include the following signals:

| Signal name | Direction | Type | Description |
|---|---|---|---|
| model_clk, rst_n | Input | Clock, | The clock and reset signals for the internal |
| | | Reset | model state machine and internal logic. |
| conf_clk, | Input | Clock, | The clock and reset signal for the configuration |
| memory_rst_n | | Reset | of the module and the communication with the |
| | | | AXI interface (eventually). |
| memory_wr_en | Input | Control | Derived by the AXI registers. That is the write |
| | | | enable signal for the soft_ram module inside the |
| | | | module. This signal is generated by the top |
| | | | module for each mRNA separately according to |
| | | | the mRNA index that is set by the AXI registers. |
| memory_wr_addr, | Input | Bus | Those are the write address and the write data |
| memory_wr_data | | | for the internal codons' memory in the soft_ram |
| | | | module. Also derived by the AXI logic. |
| req_ribo, | Output | Control | The same as in the parallel mRNA module - |
| release_ribo_to_pool, | | | those signals are used for signaling the global |
| release_ribo_from_mrna | | | arbiter. |
| grant_mrna_indx, | Input | Bus, | Those signals are derived by the global arbiter |
| grand_valid | | Control | and are used to provide the index of the mRNA |
| | | | that received a ribosome (if grant_valid is high). |
| ready_for_next_iteration | Output | Control | This signal goes high whenever the internal |
| | | | state machine of the module finishes iterating |
| | | | over all active ribosome (or when there are no |
| | | | active ribosomes). This signal is used to |
| | | | synchronize all mRNA molecules in the chip via |
| | | | the hold signal that is generated in the top module. |
| hold | Input | Control | When this signal is High, the mRNA cannot |
| | | | proceed to the next iteration. As explained, this |
| | | | signal is responsible for synchronizing the |
| | | | mRNA molecules. |
| local_time | Output | Bus | The local time of the mRNA module in |
| | | | milliseconds in real cell time. |
| proteins_counter | Output | Bus | The proteins counter of the current mRNA. |
| stop_counting | Input | Control | When the model reaches the end time, the top |
| | | | module derives 1 to this signal to freeze the |
| | | | proteins counter. |

The grant_mrna_indx and grant_valid signals replace the single input ribo_granted signal that was in the parallel mRNA to avoid a wide multiplexer. The mRNA modules derive the ribo_granted signal internally as follows:

assign ribo_granted=grant_valid & (granted_mrna_indx==P_MRNA_INDX);

Also, as can be noticed from the interface, the mRNA module is not aware of the stopping time directly, for simplicity. The only thing that is important when reaching the stopping time is to freeze the proteins counter and that is achieved using the stop_counting. The reset of the module can keep running until the next reset sequence initiated by the CPU using the AXI registers.

Reference is now made to FIG. 22, which is a high-level block diagram depicting a system 100 for whole-cell process simulations, according to some embodiments of the invention. System 100 of FIG. 22 may be the same as system 100 of FIG. 3.

As shown in FIG. 22 system 100 may include one or more hardware-implemented modules, each representing a biological entity in a simulated biological cell.

For example, system 100 may include a plurality of resource hardware modules 210 (e.g., 210A, 210B) that may represent a corresponding plurality of entities in a simulated biological cell, which may be referred to herein as “resource” entities. Additionally, system 100 may include a plurality of client hardware modules 20 (e.g., 20A, 20B) that may represent a corresponding plurality of entities in a simulated biological cell, which may be referred to herein as “client” entities.

The terms “resource” and “client” may be used herein in this context to indicate a relationship between entities in a simulated biological cell, where a simulated resource entity may be utilized by a simulated client entity to provide a service, in relation to a specific biological process.

For example, in a process of mRNA translation, a simulated client entity in a simulated biological cell may be a simulated mRNA strand, and a simulated resource entity may be a simulated ribosome, which may be utilized by the simulated mRNA strand for the purpose of mRNA translation.

In this example, system 100 may perform whole-cell simulation of a process of mRNA translation, where one or more (e.g., a plurality) of hardware resource modules 210 may represent an expected, or simulated behaviour of one or more (e.g., a plurality of) respective simulated resource entities such as ribosomes in a biological cell. Additionally, one or more (e.g., a plurality) of hardware client modules 20 may represent an expected behaviour of one or more (e.g., a plurality) of respective simulated client entities such as mRNA strands in the simulated biological cell. In this example, the simulated ribosomes may be regarded as providers of a service (e.g., codon translation) to the simulated client mRNA strands.

The one or more (e.g., plurality of) hardware resource modules 210 (e.g., representing simulated ribosomes) may produce a first predicted or simulated value 210′, referred to herein as a resource behaviour value 210′. Resource behaviour value 210′ may represent an expected behaviour, or an aspect of behaviour of at least one corresponding simulated resource entities (e.g., ribosomes) in the simulated biological cell.

In the example of mRNA translation, the predicted or simulated resource behaviour value 210′ may include one or more values pertaining to the process of mRNA translation.

For example, resource behaviour value 210′ may be, or may include resource status, indicating whether a corresponding simulated ribosome is either (i) currently associated to a pool of free ribosomes, or (ii) occupied by, associated to or allocated to a specific simulated mRNA strand of the simulated biological cell.

In another example, resource behaviour value 210′ may be, or may include a duration of translation of at least one codon or codon type by a corresponding simulated ribosome of the simulated biological cell.

In another example, resource behaviour value 210′ may be a duration of the corresponding simulated resource (e.g., ribosome) to process a predetermined number of simulated codons, and/or an initiation rate, e.g., the time it takes for a simulated ribosome that corresponds to the resource module 210 to initiate actual translation of a simulated mRNA strand (e.g., after being allocated to that strand).

In another example, resource behaviour value 210′ may be a ribosome footprint, representing a number of simulated codons that the corresponding simulated ribosome may handle, or translate concurrently.

In yet another example, resource behaviour value 210′ may be a diffusion delay value, representing a time it takes for the corresponding simulated ribosome, after finishing translation of one mRNA strand and/or after being de-allocated from one mRNA strand, to become available for translating another simulated mRNA strand.

The one or more (e.g., plurality of) hardware client modules 20 (e.g., representing simulated client mRNA strands) may produce one or more predicted or simulated interaction values 20′. Each interaction value 20′ may represent an aspect of interaction of the corresponding simulated client entity (represented by a hardware client module 20) in the simulated biological cell with at least one resource entity (represented by a resource module 210) in the simulated biological cell.

In the example of mRNA translation, such predicted or simulated interaction value 20′ may include, for example a state of activity of one or more simulated ribosomes allocated to the corresponding simulated mRNA strand. This state of activity may be, for example (i) an inactive state, in which translation codons is currently not performed and (ii) an active state, in which translation of a simulated codon is currently performed.

Additionally, or alternatively, interaction value 20′ may include, for example a number of ribosomes (represented by hardware resource modules 210) that are applied to, allocated to, or reside on a specific simulated mRNA strand entity (represented by a hardware client module 20), and/or a number of active ribosomes (represented by resource modules 210) that are currently performing translation of the corresponding mRNA strand entity (represented by client module 20).

Additionally, or alternatively, interaction value 20′ may include a location of one or more (e.g., each) simulated ribosome (represented by resource modules 210) on the simulated mRNA strand entity (represented by client module 20).

Additionally, or alternatively, interaction value 20′ may include one or more codon indices, representing a codon (or location of a codon) that is being translated by a simulated ribosome (represented by resource modules 210) on the corresponding simulated mRNA strand (represented by client module 20).

Additionally, or alternatively, interaction value 20′ may include statistic information regarding a number of completed translations of the mRNA strand entity (client module 20), such as a quantity of protein molecules generated by the relevant mRNA strand; statistic information regarding the state of activity of ribosomes (hardware client module 20) on the represented mRNA strand (client module 20); statistic information regarding occurrence of “traffic jams” of ribosomes on the represented mRNA strand; a length of each client entity (e.g., number of codons of each mRNA strand, reflecting the expected time of translation), and the like.

According to some embodiments, system 100 may include a hardware module, referred to herein as a free resources' counter module 40 (or counter 40, for short). Counter 40 may be configured to count, or keep track of free resource entities in the simulated biological cell. In the example of mRNA translation, the plurality of resource modules 210 may represent a corresponding plurality of free resource entities (e.g., ribosomes) in the simulated biological cell. Each of the resource modules 210 may be allocated, or assigned to a specific client entity 20, representing an mRNA strand in the simulated biological cell.

Hardware free resources' counter module 40 may be initialized to the total number of resource modules 210 (e.g., the total number of ribosomes in the cell), and may keep track of the current number of free (e.g., unallocated, or unassigned) resource modules 210. In the example of mRNA translation, the unassigned resources may be ribosomes (represented by resource modules 210) that are currently not attached to mRNA strands (represented by client modules 20).

In the example of simulated mRNA translation, at each time point a resource module 210 (e.g., a simulated ribosome) from a pool of free resource modules 210 can initiate translation of only one client 20 (e.g., a simulated mRNA strand), or remain in the pool of free resources (e.g., ribosomes) 210.

Additionally, or alternatively, at each time point a resource module 210 (e.g., a simulated ribosome) which was allocated to a client 20 (e.g., a simulated mRNA strand), to initiate translation may move to the next codon, according to a predefined set of rules. For example, a ribosome-representing resource module 210 may move to the next codon (a) if it is not blocked by a ribosome downstream of it; and (b) after it has waited on the current codon for at least the decoding time related to the codon.

Additionally, or alternatively, at each time point, a resource module 210 (e.g., a simulated ribosome) that is located at a final codon (e.g., at the end of the mRNA strand) may terminate the translation process, and move to the pool of free resource modules 210 (e.g., free ribosomal pool) after waiting on the final codon for at least the decoding time related to that codon.

It may be appreciated by a person skilled in the art that such hardware-based simulation of a biological process, may be performed in parallel, e.g., having more than one resource module 210 and/or client module 20 active at the same time. Embodiments of the invention may thus produce simulation results of biological processes in a fraction of the time that would be required for equivalent, software-based systems of biological process simulation.

According to some embodiments, system 100 may include, or may employ a hardware arbitration module, denoted herein as a global arbiter module 30.

Arbiter module 30 may be configured to allocate one or more resource (e.g., ribosome) hardware modules 210 of the plurality of resource hardware modules 210 to at least one client (e.g., mRNA) hardware module 20 of the plurality of client hardware modules 20.

For example, one or more hardware client representation modules 20 may communicate an allocation request 21 to arbiter 30, to request allocation of a resource entity (e.g., represented by resource representation module 210). In the example of mRNA translation, the one or more hardware client representation modules 20 may represent mRNA strands, and may request allocation 21 of a resource entity 210 (e.g., a simulated ribosome) to the relevant mRNA strand.

In a complementary example, one or more hardware client representation modules 20 may communicate a deallocation request 22 to arbiter 30, to free a resource entity. In the example of mRNA translation, the one or more hardware client representation modules 20 may represent mRNA strands, and may request deallocation 21 of a resource entity 210 (e.g., ribosome) to free the simulated ribosome from to the relevant simulated mRNA strand.

Arbiter 30 may collaborate with counter 40 to manage the access requests (e.g., 21, 22) of hardware client representation modules 20 (e.g., representing mRNA strands) to the simulated pool of free resource entities (e.g., ribosomes).

For example, arbiter 30 may assign or allocate free resource representation modules 210 (e.g., representing simulated free ribosomes) to client representation modules 20 (e.g., representing simulated mRNA strands) with uniform probability. Arbiter 30 may subsequently update counter 40 on this allocation, so as to maintain a correct count of available resource representation modules 210.

In a complementary example, arbiter 30 may deallocate or free resource representation modules 210 (e.g., representing ribosomes) from client representation modules 20 (e.g., representing mRNA strands). Arbiter 30 may subsequently update counter 40 on this deallocation, to maintain a correct count of available resource representation modules 210.

According to some embodiments, arbiter module 30 may subsequently produce or predict one or more simulated arbitration values 30′ based on this allocation. Arbitration values 30′ may each represent an aspect of allocation of simulated resource entities to simulated client entities in the simulated biological cell. In other words, arbitration values 30′ may represent arbitration of interactions between the plurality of resource entities of the simulated biological cell, and the plurality of client entities in the simulated biological cell.

For example, predicted or simulated arbitration value 30′ may include, for example an overall number of simulated resource entities (e.g., ribosomes, represented by resource modules 210) in the simulated biological cell, an overall number of simulated client entities (e.g., mRNA strands, represented by client modules 20) in the simulated biological cell; a number of free, or available resource entities (e.g., ribosomes) in the simulated biological cell, a number of client entities (e.g., mRNA strands, represented by client modules 20) that are currently allocated simulated resource (e.g., ribosome) entities in the simulated biological cell, a number of resource entities that are allocated to each client entity (e.g., the number of simulated ribosomes that are associated to each mRNA strand), a number of simulated mRNA strands that are currently being translated by allocated simulated ribosomes.

According to some embodiments, system 100 may include an analysis module 60. Analysis module 60 may include one or more processing units, such as Zynq processors 110 of FIG. 8, configured to calculate a simulated product value 60′. Product value 60′ may represent, or pertain to, a product of a biological process in the simulated biological cell.

According to some embodiments, analysis module 60 may calculate simulated product value 60′ based on one or more of: (a) the predicted or simulated resource behaviour values 210′; (b) the predicted or simulated interaction values 20′; and (c) the predicted or simulated arbitration values 30′. Additionally, or alternatively, analysis module 60 may calculate or provide product value 60′ as an outcome of the simulated whole-cell process described herein, based on (a) the predicted or simulated resource behaviour values 210′; (b) the predicted or simulated interaction values 20′; and/or (c) the predicted or simulated arbitration values 30′.

It may be appreciated that the variety of applications of whole-cell process simulation is very large, and calculation of simulated product value 60′ may be application-specific. Accordingly, the number of possible calculations of simulated product value 60′ may be large as well.

Pertaining to the non-limiting example where the biological process is one of simulating a whole-cell mRNA translation process, by which simulated mRNA strands are translated by the plurality of ribosomes to produce simulated protein molecules: In this example, simulated product value 60′ may be, or may include statistic information regarding a simulated quantity of proteins generated by the simulated cell, e.g., within a given timeframe, by the process of translation. In such embodiments, analysis module 60 may be, or may include a protein counter module 60 as elaborated herein. Additionally, or alternatively, analysis module 60 may be configured to read all protein counters 27 from all mRNA modules 20, to provide a simulated product value 60′ that is an accumulated quantity of simulated, produced protein molecules in the simulated cell.

As a simplified example, arbitration values 30′ may include (i) a number of ribosomes in the simulated cell and (ii) a number of simulated mRNA strands of the specific protein in the simulated cell, resource behaviour values 210′ may include (iii) a time that it takes for a simulated ribosome to translate a simulated mRNA strand of a specific protein, and interaction value 20′ may include (iv) a number of active simulated ribosomes that may be applied to the specific simulated mRNA strand type. System 100 may perform whole-cell simulation of mRNA translation, based on (i)-(iv) above, where each client (mRNA) representation module 20 may count the number of simulated protein molecules that are produced via translation of the respective simulated mRNA strand. Analysis module 60 may thus communicate with each of the plurality of client representation modules 20 (e.g., representing simulated mRNA strands) to calculate (e.g., by one or more processors 110) a required statistic. Such statistic data may be, for example a quantity of a product (e.g., protein) of the simulated biological process, a mean value and/or standard deviation value of simulated protein molecules, a rate of production of simulated protein molecules that are produced by the simulated cell, and the like.

In another example, and as elaborated herein (e.g., in relation to FIG. 23) system 100 may perform whole-cell simulation of other biological processes, including for example DNA transcription. In this example, system 100 may simulate whole-cell DNA transcription based on one or more of: (a) the predicted or simulated resource behaviour values 210′; (b) the predicted or simulated interaction values 20′; and (c) the predicted or simulated arbitration values 30′. Analysis module 60 may calculate or provide product 60′ as a product of the simulated DNA transcription process (e.g., a simulated quantity of produced RNA molecules) as a result of this simulation.

Additionally, or alternatively, analysis module 60 may calculate or produce product 60′ as a selection of an optimal client entity, given a predefined organism and a predefined objective.

For example, given the predefined organism (e.g., E. Coli) parameters of system 100 (e.g., codon decoding rates, initiation rates, mRNA levels of each gene, and the number of ribosomes) may be configured in hardware, as elaborated herein. The predefined objective may include for example, a decrease of the number of simulated ribosomes on a simulated mRNA strand, or obtaining a high rate of protein production. In such embodiments, system 100 may simulate a process of mRNA translation and protein generation based on the preconfigured system parameters. Client modules 20 may represent functionally similar simulated mRNA strands (e.g., SNPs), that may produce functionally similar simulated proteins. Based on this simulation, analysis module 60 may identify a simulated mRNA strand that corresponds to the predefined objective (e.g., highest rate of protein production). Analysis module 60 may thus calculate or produce product 60′ as a selected simulated mRNA strand among the plurality of functionally similar simulated mRNA strands, based on the whole-cell simulation as elaborated herein.

It may be appreciated that embodiments of the invention may include a practical application for engineering of biological cells. Pertaining to the example of optimal mRNA strand selection presented above, product 60′ may be used for applications of bioengineering, to generate real-world genetic entities (e.g., Genes, DNA sequences, RNA sequences and the like) as known in the art, where the generated genetic entities correspond to the selected client (e.g., mRNA) biological entities.

As elaborated herein (e.g., in relation to FIG. 5), system 100 may include one or more client (e.g., mRNA) representation modules 20 (e.g., 20A) of a first type, referred to herein as “parallel mRNAs” 20A. Additionally, or alternatively, and as elaborated herein (e.g., in relation to FIG. 6), system 100 may include one or more client (e.g., mRNA) representation modules 20 (e.g., 20B) of a second type, referred to herein as “iterative mRNAs” 20B.

According to some embodiments, the one or more parallel mRNA modules 20A may each include, or be associated with one or more (e.g., a plurality of) hardware resource representation modules 210A (e.g., representing ribosome entities in a simulated cell). As elaborated herein, each hardware resource representation module 210A may be, or may include a hardware state machine 20SM, that may simulate the behavior of resource entities (e.g., ribosomes) in a simulated biological cell.

State machine 20SM may be responsible for: (a) communicating with global arbiter module 30, and (b) activation and/or deactivation of the hardware resource representation modules 210A. Pertaining to the mRNA translation example, state machine 20SM may activate or deactivate resource modules 210A according to messages from global arbiter module 30, to respectively represent translation, or cessation of translation of mRNA strands in a simulated cell.

According to some embodiments, once activated, each of the one or more hardware resource representation modules 210A may operate autonomously and in parallel to each other. It may be appreciated that such behaviour may mimic that of resource entities (e.g., ribosomes) in a biological cell. In a similar manner, the one or more parallel hardware client representation modules 210A may operate autonomously and in parallel, to mimic the behaviour of client entities (e.g., mRNA strands) in a biological cell.

Additionally, or alternatively, the one or more iterative client modules 20 (e.g., 20B, representing mRNA strands) may each include, or be associated with, a hardware resource (e.g., ribosome) representation module 210B. Hardware resource representation module 210B may globally represent the resource entities (e.g., ribosomes) that are associated with, or allocated to the relevant iterative mRNA module 20B.

For example, hardware resource (e.g., ribosome) representation module 210B may include a data structure such as a FIFO or queue 210FF. FIFO 210FF may maintain the current state of a plurality of (e.g., all) resource entities (e.g., ribosomes) that are currently allocated to a specific client entity (e.g., mRNA strand) that is represented by the relevant iterative client (e.g., mRNA) module 20B.

In other words, and pertaining to the mRNA translation example, FIFO 210FF may include a plurality of entries, each representing a specific simulated ribosome, and a specific simulated mRNA strand, to which that simulated ribosome is allocated. Each entry of FIFO 210FF may include information such as a current state (e.g., active/inactive) of the relevant simulated ribosome; a current index (e.g., an identification of a codon) of the relevant simulated ribosome on the simulated mRNA strand; remaining translation time of the current codon, and the like.

In such embodiments, iterative client (e.g., mRNA) module 20B may traverse, or iterate over all entries in FIFO 210FF to manage the state of the currently active simulated ribosomes. Since each simulated client entity (e.g., mRNA), represented by a specific iterative client module 20B can be allocated a different number of ribosomes, this technique may require synchronization between client (e.g., mRNA) modules 20, to avoid having client (e.g., mRNA) modules 20 with a small number of resources (e.g., ribosomes) from being translated faster than client (e.g., mRNA) modules 20 with a larger number of resources (e.g., ribosomes), due to the overall number of calculations to be done in each iteration over FIFO 210FF.

Embodiments of the invention may leverage a tradeoff between characteristics of parallel client (e.g., mRNA) modules 20A and iterative client (e.g., mRNA) modules 20B to provide further improvement in technology of biochemical process simulation: On one hand, due to the synchronization overhead, iterative client (e.g., mRNA) modules 20B may operate slower (e.g., 1-2 orders of magnitude slower) than parallel client (e.g., mRNA) modules 20A. On the other hand, iterative mRNA modules 20B may contain a single FIFO 210FF representing a plurality of resource (e.g., ribosome) instances (rather than a plurality of hardware instances, each representing a single ribosome), and may therefore consume less hardware resources (e.g., silicon area, power, etc.) in comparison to parallel mRNA modules 20A.

According to some embodiments, system 100 may include a combination of the two models of client representations modules (e.g., parallel mRNA modules 20A and iterative mRNA modules 20B), according to a predefined application.

For example, system 100 may be employed to select an optimal mRNA mutation or Single Nucleotide Polymorphism (SNP) among a plurality of mRNA mutations. For example, system 100 may be employed to detect mRNA strands that should be used to optimize a certain synthetic biology objective, e.g., produce the largest quantity of protein molecules and/or produce a most noticeable phenotypic effect within a predefined time period. System 100 may start by running wide searches over large quantities of mRNA mutations by using an iterative client (e.g., mRNA) module 20B. System 100 may select potential mRNA candidates for further analysis, and proceed to analyze the selected subset of mRNA strands using parallel mRNA modules 20A. In this context, the term “candidate” may be used to indicate an mRNA strand that may have the desired effect on the objective. In that way, system 100 may be able to focus on the most interesting mRNAs and examine 1-2 orders of magnitude more mutations than would have been possible with iterative mRNA module 20B in the same amount of time.

Reference is now made to FIG. 23, which is a high-level block diagram depicting a system 100 for whole-cell process simulations, according to some embodiments of the invention. According to some embodiments, system 100 of FIG. 23 may be the same as system 100 of FIG. 3, and/or system 100 of FIG. 22. Accordingly, system 100 of FIG. 23 may include the same modules and functions as elaborated herein (e.g., in relation to system 100 of FIG. 3, and/or system 100 of FIG. 22).

It may be appreciated that uniform arbitration may be needed to simulate a variety of whole-cell biochemical processes (e.g., not just processes of mRNA strand translation). Such processes may include a cascade of sub-processes, which may, or may not be co-dependent.

For example, and as elaborated in the non-limiting example of FIG. 23, system 100 may employ uniform arbitration to simulate or model whole-cell biochemical processes of protein generation. This process may be divided to a first sub-process that may simulate utilization of a first pool of resources (e.g., simulated ribosomes) and a second sub-process that may simulate utilization of a second pool of resources (e.g., a cell's pool of transfer RNA (RNA)).

As known in the art, a biological cell typically has 61 types of tRNAs molecules. Each cell has a limited amount or quantity of each type of tRNA molecules. In a similar manner as elaborated herein in relation to ribosomes, a pool of tRNA molecules of a simulated cell may be shared among all the ribosomes and may be received by, or allocated to ribosomes in a stochastic manner. In that sense, tRNA molecules may be regarded as resource entities in the simulated cell, and the ribosomes may be regarded as client entities in the simulated cell. As such, the tRNA pools in the simulated cell can be modeled using hardware arbiter modules 30 and free resource counters 40, which may be implemented in the same technique as arbiter modules 30, and free resource counters 40, as elaborated herein (e.g., in relation to ribosome resources of FIG. 22).

According to some embodiments, system 100 may include 61 hardware-based tRNA arbiters 30 and 61 hardware-based free resource (tRNA) counters 40, corresponding to the 61 types of tRNAs in the simulated cell. For the purpose of clarity, the resource (ribosomes) arbiter module 30 of FIG. 22 is denoted herein as arbiter module 30A; the free resource (ribosomes) counter of FIG. 22 is denoted herein as counter 40A; the plurality (e.g., 61) of tRNA arbiter modules 30 are denoted herein as arbiter modules 30B; and the plurality (e.g., 61) of free resource (tRNA) counters are denoted herein as counters 40B.

Additionally, in the example of FIG. 23, resource (ribosomes) management modules such as state machine 20SM of parallel mRNA modules 20A may be adapted to support an additional state, in which the ribosomes resource representation modules 210 await an assignment of a specific simulated tRNA molecule of the plurality (e.g., 61) of tRNA molecules that is suitable for the current codon type.

Additionally, as shown in the example of FIG. 23, system 100 may include a hardware-based interconnection layer module 50, which may perform a function of interconnection between the two underlying processes: e.g., mRNA translation by ribosome resources, and protein generation by tRNA resources.

For example, interconnection layer module 50 may be configured to provide resources to clients based on the interactions with the various relevant arbiters. In other words, interconnection layer module 50 may be responsible for providing a mesh access for the shared, simulated resource entities in the simulated cell to/from all clients. According to some embodiments, interconnection layer module 50 may be implemented by an on-chip interconnection module or interconnection “fabric” such as AXI.

It may be appreciated that system 100 may be adapted or modified to provide efficient simulation of a variety of whole-cell processes, and may not necessarily be limited to the examples of mRNA translation and protein generation, as brought herein.

For example, system 100 may employ uniform arbitration as elaborated herein to simulate or model whole-cell biochemical processes such as transcription of genetic material. It may be appreciated that the process of transcription may be modelled very similarly to that of mRNA translation, as elaborated herein.

Thus, system 100 as presented here may be used in the capacity of whole-cell genetic transcription modelling, with the appropriate modification of parameters. For example, in order to simulate DNA transcription (a) instead of using resource representation modules 210 that model, or represent ribosomes, system 100 may include resource representation modules 210 that model, or represent simulated RNA polymerase molecules in the simulated biological cell; and (b) instead of using client representation modules 20 that represent mRNA strands, system 100 may include client representation modules 20 that model, or represent simulated genes (e.g., the part of genetic material such as RNA or DNA to be transcribed) in the simulated biological cell.

Additional examples for applying system 100 for whole-cell biochemical process simulation may include, for example modelling of competition of mRNA strands on miRNA molecules, modelling of competition of genes on transcription factors, and the like.

As known in the art, currently available solutions for hardware acceleration target runtime bottlenecks of given software algorithms and model them in hardware. Such methods produce solutions that are mainly operated by a software, with bottlenecks implemented in hardware that is commonly referred to as an accelerator. It has been experimentally shown that the current paradigm, based on hardware-accelerated software proved irrelevant for the purpose of whole-cell process modelling (e.g., modelling of mRNA translation) in the Totally Asymmetric Simple Exclusion Process (TASEP) approach, which is a stochastic simulation technique commonly used for describing ribosomal movement.

As elaborated herein, the modules of system 100 (e.g., modules 20, 210, 30, 40 and 60) may be referred to herein as hardware modules, in a sense that they may be at least partially implemented as programmable logic on a hardware chip, such as an FPGA or an ASIC chip.

In other words, a system 100 for whole-cell simulation may be, or may include a system-on-chip (SoC) implementation. Such an SoC-implemented system 100 may include a plurality of hardware modules or circuits. This plurality of hardware modules or circuits may include a plurality of client representation modules 20, each representing a client entity in the simulated biological cell. Additionally, the plurality of hardware modules may include a plurality of resource representation modules 20, each representing a resource entity in the simulated biological cell, as elaborated herein. Additionally, the plurality of hardware modules may include one or more arbiter modules 30, each representing a process of arbitration in the simulated biological cell, as elaborated herein. Additionally, the plurality of hardware modules may include one or more counter modules, each representing a pool of resource entities in the simulated biological cell, as elaborated herein. As elaborated herein, the plurality of hardware modules on the SoC may be configured to collaborate in parallel, thus obtaining parallel simulation of behaviour of individual client entities and/or resource entities in the simulated biological cell.

System 100 may thus include an improvement of efficiency over currently available software-based and/or hardware accelerated methods of biochemical process simulation. As elaborated herein, system 100 may be implemented in an innovative approach, where all relevant cellular entities (e.g., mRNA strands, ribosome pool, singular ribosomes, etc.) are represented or simulated by dedicated hardware modules (e.g., 20, 40, 210, respectively). Each of the dedicated hardware modules may be designed according to specific properties of each cellular entity. By doing so, system 100 may provide a fully autonomous, hardware model of a simulated biological cell, with all relevant biological entities running in parallel (e.g., irrespective of other biological entities) and autonomously (e.g., unbound by serial propagation of software tasks and processes).

Additionally, system 100 may include an improvement of visibility over currently available software-based and/or hardware-based accelerated methods of biochemical process simulation. As system 100 may be implemented by hardware modules, the communication processes that occur between entities in the simulated cell model of system 100 may be captured and analyzed. Therefore, system 100 may provide a unique ability to extract meaningful statistics and insights regarding internal, time-dependent processes in a real biological cell. In other words, system 100 may allow understanding of intracellular processes with high resolution, facilitating future engineering of cellular components.

Additionally, system 100 may include an improvement of compactness, cost and personalization over currently available software-based and/or hardware accelerated methods of biochemical process simulation. For example, currently available systems for biochemical process simulation require high-end software computing and processing devices. As system 100 may be implemented on a relatively small piece of hardware (e.g., an FPGA device), without need for such high-end software processing devices, embodiments of system 100 may allow personalized analysis of cellular processes, such as monitoring and analysis of expression of individual genes.

Additionally, system 100 may include an improvement of accuracy over currently available software-based and/or hardware accelerated methods of modelling whole-cell biochemical processes. For example, as known in the art, current methods for hardware arbitration may include iteration over endpoints (e.g., entities of hardware resources), sequentially or with a fixed priority, to schedule access to, or allocation of these hardware resources. As elaborated herein, global arbiter module 30 may be configured to perform arbitration of shared hardware resources, such as the shared pool of resource modules 210 (e.g., representing ribosome entities in a simulated cell). However, it has been experimentally shown that for the purpose of whole-cell process simulation, the existing techniques are not relevant, since the connections between resource entities (e.g., ribosomes) and client entities (e.g., mRNA strands) in the cell happen randomly, in a stochastic manner. According to some embodiments of the invention, global arbiter module 30 may be configured to arbitrate, or share the common resources 210 between clients 20 in a stochastic manner. Therefore, arbiter hardware module 30 may be configured to allocate the plurality (e.g., one or more) resource (e.g., ribosomes) hardware modules 210 to the plurality of clients (e.g., mRNA) modules 20 with uniform probability. Therefore, system 100 may model the process of resource allocation that takes place in a simulated biological cell in a manner that is more biologically accurate.

The following is the list of parameters for the automatically generated top chip module.

| Parameter | Description |
|---|---|
| P_NUM_MRNAS | This parameter dictates the amount |
| | of mRNA molecules instantiated in the |
| | top module. |
| P_GLOBAL_TIME_WIDTH | The bit width of the local timer of each |
| | mRNA molecule. |
| P_FREE_RIBOS_COUNTER_WIDTH | The width of the ribosomes counter. |
| P_GENERATED_PROTS_COUNTER_WIDTH | The width of the local proteins counter for |
| | each mRNA molecule. |

### Uniform Arbiter—Parameters

| Parameter | Description |
|---|---|
| P_FREE_RIBOS_COUNTER_WIDTH | The width of the free ribosomes' counter in the cell. |
| P_MUX_WIDTH_LVL½ | As explained in the pipelined multiplexer module - |
| | those parameters define the width of the first and |
| | second level multiplexers. |
| P_MRNA_INDX_WIDTH | The width of the outputted mRNA index. The output |
| | of the module contains the index of the selected |
| | mRNA module. |

### Hardware Ribosome Module

| Parameter | Description |
|---|---|
| P_MRNA_LENGTH | The length of the mRNA molecule (in codons). |
| P_CODON_INDX_WIDTH | The width of the index to the codons in the specific mRNA. |
| | This value depends on the length of the mRNA and therefore |
| | parametric. |
| P_CODON_DELAY_WIDTH | The width of the translation delay timer of the codons. |
| P_RIBO_MIN_DISTANCE | The minimal distance (in codons) that should be kept |
| | between two consecutive ribosomes). |
| P_RIBO_ALLOC_DELAY | The allocation time for the specific mRNA. |
| P_RIBO_INDX | The index of the current ribosome in the hardware ribosomal |
| | chain. This value is used to check if the current served |
| | ribosome by the data arbiter is the local ribosome. |
| P_MAX_ACTIVE_RIBOS | Number of hardware ribosomes in the current mRNA. |

mRNA Module—Interface and Parameters

The parameters P_CODON_INDX_WIDTH, P_CODON_DELAY_WIDTH, P_RIBO_MIN_DISTANCE, P_MAX_ACTIVE_RIBOS, P_RIBO_ALLOC_DELAY, P_MRNA_LENGTH, P_DEFUSION_TIME and P_DEFUSION_ITERATION_TIME are the same as before. Those are the new parameters introduced by this module:

| Parameter | Description |
|---|---|
| P_MRNA_INDX | The index of the current mRNA module. This |
| | is used when the arbiter outputs the index of the |
| | mRNA that is currently served. |
| P_MRNA_INDX_WIDTH | The width in bits of the mRNA index param. |
| P_CODON_WIDTH | The width of the codons' code. |
| P_DELAY_WIDTH | The maximal width of the ribosomes' timer that |
| | is used for the allocation delay and for the |
| | codons' delay. |
| P_ALLOC_DELAY_WIDTH | The width of the allocation delay. |
| P_RIBO_FIFO_ADDR_WIDTH | The address width of the FIFO used to store the |
| | ribosomes' state. |
| P_LOCAL_TIME_WIDTH | The width of the local time counter. |
| P_CODONS_MEM_FILE | Path to the .mem file used to initialize the |
| | lut_rom module with the codons delay values. |
| P_CODONS_DELAY_FILE | Path to the .mem file used to initialize the |
| | soft_rom module with the specific mRNA's |
| | codons' list. |
| P_GENERATED_PROTS_CNT_WIDTH | The width of the local protein counter. |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

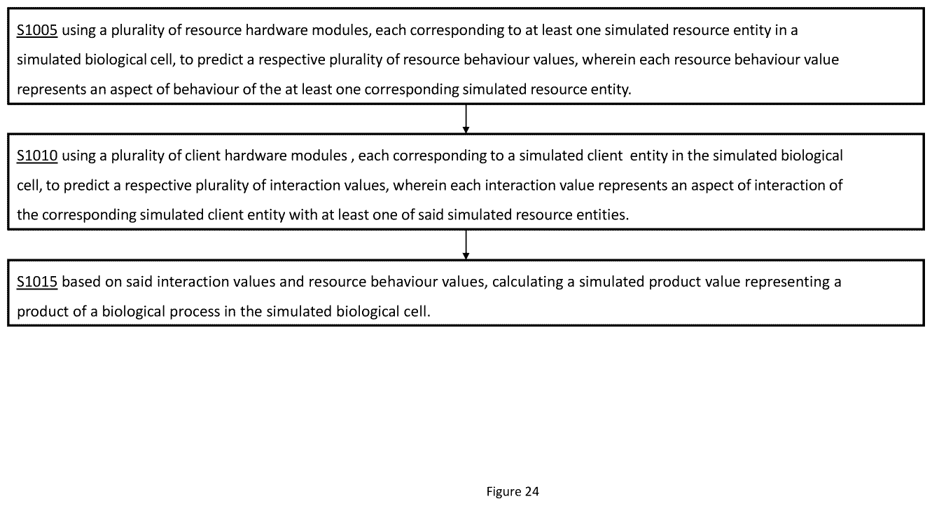

Reference is now made to FIG. 24, which is a flow diagram depicting an example of a method of whole-cell process simulation by one or more dedicated, hardware-implemented electrical circuits, according to some embodiments of the invention.

As shown in step S1005, embodiments of the method may include using a plurality of hardware-implemented electrical circuits referred to herein as resource hardware modules (e.g., resource modules 210 or ribosome modules 210), each corresponding to, or representing at least one simulated resource entity (e.g., a ribosome) in a simulated biological cell, to predict a respective plurality of resource behaviour values (e.g., 210′). Each resource behaviour value 210′ may represent an aspect of behaviour of the at least one corresponding simulated resource entity, as elaborated herein.

As shown in step S1010, embodiments of the method may include using a plurality of hardware-implemented electrical circuits referred to herein as client hardware modules (e.g., client modules 20, or mRNA modules 20), each corresponding to a simulated client entity (e.g., an mRNA strand) in the simulated biological cell, to predict a respective plurality of interaction values 20′. Each interaction value 20′ may represent an aspect of interaction of the corresponding simulated client entity (e.g., mRNA strand) with at least one of said simulated resource entities (e.g., ribosomes).

As elaborated herein, the plurality of resource hardware modules 210 and the plurality of client hardware modules 20 may be implemented, at least in part as programmable logic on a dedicated hardware electrical circuit such as an FPGA chip or an ASIC chip. As shown in step S1015, embodiments of the method may include calculating, by at least one processor embedded in the dedicated hardware electrical circuit, a simulated product value representing a product of a biological process in the simulated biological cell, based on said interaction values and/or resource behaviour values, as elaborated herein.

1. A method of whole-cell process simulation, the method comprising:
using a plurality of resource hardware modules, each corresponding to at least one simulated resource entity in a simulated biological cell, to predict a respective plurality of resource behaviour values, wherein each resource behaviour value represents an aspect of behaviour of the at least one corresponding simulated resource entity;
using a plurality of client hardware modules, each corresponding to a simulated client entity in the simulated biological cell, to predict a respective plurality of interaction values, wherein each interaction value represents an aspect of interaction of the corresponding simulated client entity with at least one of said simulated resource entities; and
based on said interaction values and resource behaviour values, calculating a simulated product value representing a product of a biological process in the simulated biological cell.

2. The method of claim 1, further comprising:
using an arbitration hardware module to allocate one or more resource hardware modules of the plurality of resource hardware modules to at least one client hardware module of the plurality of client hardware modules;
based on said allocation, predicting one or more arbitration values, each representing an aspect of allocation of simulated resource entities to simulated client entities in the simulated biological cell; and
calculating the simulated product value further based on said one or more arbitration values.

3. The method according to claim 2, wherein at least one of the resource hardware modules, client hardware modules and arbitration hardware modules are at least partially implemented as programmable logic on a hardware electrical circuit, selected from a list consisting of a Field Programmable Gate Array (FPGA) chip and an Application Specific Integrated Circuit (ASIC) chip.

4. The method according to claim 2, wherein the simulated resource entities are simulated ribosomes of the simulated biological cell, and wherein the simulated client entities are simulated mRNA strands of the simulated biological cell.

5. The method of claim 4, wherein the resource behaviour value comprises a resource status, indicating whether a corresponding simulated ribosome is either (i) currently associated to a pool of free ribosomes, or (ii) allocated to a simulated mRNA strand of the simulated biological cell.

6. The method of claim 5, wherein the resource behaviour value is further selected from a list consisting of: a duration of translation of at least one simulated codon or codon type by the corresponding simulated ribosome; a duration of the corresponding simulated ribosome to process a predetermined number of simulated codons; an initiation rate representing a time it takes for the corresponding simulated ribosome to initiate translation of a simulated mRNA strand; a ribosome footprint, representing a number of simulated codons that the corresponding simulated ribosome may handle concurrently; and a diffusion delay, representing a time it takes for the corresponding simulated ribosome, after finishing translation of one mRNA strand, to become available for translating another simulated mRNA strand.

7. The method according to claim 4, wherein the interaction value represents a state of activity of one or more simulated ribosomes allocated to the corresponding simulated mRNA strand, said state of activity selected from (i) inactive state, and (ii) active state, in which translation of a simulated codon is currently performed.

8. The method according to claim 7, wherein the interaction value is further selected from a list consisting of: a number of simulated ribosomes that are applied to the corresponding simulated mRNA strand; a number of active simulated ribosomes, that are currently performing translation of the corresponding simulated mRNA strand; a location of one or more simulated ribosomes on the corresponding simulated mRNA strand; and a codon index, representing a codon that is being translated by a simulated ribosome on the corresponding simulated mRNA strand.

9. The method according to claim 4, wherein the arbitration values represent aspects of allocation of simulated ribosomes to simulated mRNA strands in the simulated biological cell, said aspects selected from a list consisting of: an overall number of simulated ribosomes in the simulated biological cell; an overall number of simulated mRNA strands in the simulated biological cell; a number of ribosomes in the simulated biological cell that are available for mRNA translation; a number of simulated mRNA strands that are currently allocated to simulated ribosomes; a number of simulated mRNA strands that are currently being translated by allocated simulated ribosomes; and a number of simulated ribosomes that are allocated to each simulated mRNA strand.

10. The method according to claim 4, wherein the biological process is a process of translation of the simulated mRNA strands by the plurality of simulated ribosomes, and wherein the simulated product value is a simulated quantity of protein molecules, produced in said process of translation.

11. The method according to claim 2, wherein the arbitration hardware module is configured to allocate the one or more resource hardware modules to the plurality of client hardware modules with uniform probability.

12. The method according to claim 1, wherein the plurality of client hardware modules comprises at least one client hardware module of a first client type and one or more second client hardware modules of a second client type, and wherein predicting an interaction value comprises
using the at least one client hardware module of the first client type to identify a subset of simulated client entities in the simulated biological cell, characterized by a first desired objective; and
using the one or more client hardware modules of the second client type, to select a simulated client entity in the simulated biological cell, characterized by a predefined synthetic biological objective.

13. The method according to claim 1, wherein the biological process is a process of gene transcription, and wherein the plurality of resource hardware modules represent simulated RNA polymerase molecules, and wherein the plurality of client hardware modules represent simulated genes in the simulated biological cell.

14. A system for whole-cell process simulation, the system comprising:
a plurality of resource hardware modules, each representing at least one simulated resource entity in a simulated biological cell, and configured to predict a respective plurality of resource behaviour values, wherein each resource behaviour value represents an aspect of behaviour of the at least one corresponding simulated resource entity;
a plurality of client hardware modules, each representing a simulated client entity in the simulated biological cell, and configured to predict a respective plurality of interaction values, wherein each interaction value represents an aspect of interaction of the corresponding simulated client entity with at least one of said simulated resource entities; and
at least one processor, configured to calculate a simulated product value, representing a product of a biological process in the simulated biological cell, based on said interaction values and resource behaviour values.

15. The system of claim 14, further comprising an arbitration hardware module, configured to allocate one or more resource hardware modules of the plurality of resource hardware modules to at least one client hardware module of the plurality of client hardware modules, and wherein the at least one processor is further configured to:
predict one or more arbitration values based on said allocation, wherein each arbitration value representing an aspect of allocation of simulated resource entities to simulated client entities in the simulated biological cell; and
calculate the simulated product value further based on said one or more arbitration values.

16. The system according to claim 14, wherein at least one of the resource hardware modules, client hardware modules and arbitration hardware modules are at least partially implemented as programmable logic on a hardware electrical circuit, selected from a list consisting of an FPGA chip and an ASIC chip.

17. The system according to claim 14, wherein the simulated biological process is a process of mRNA translation, wherein the simulated resource entities are simulated ribosomes of the simulated biological cell, and wherein the simulated client entities are simulated mRNA strands of the simulated biological cell.

18. The system according to claim 14, wherein the biological process is a process of gene transcription, and wherein the plurality of resource hardware modules represent simulated RNA polymerase molecules, and wherein the plurality of client hardware modules represent simulated genes in the simulated biological cell.
